(19) United States
(12) Patent Application Publication
(10) Pub. No.: US 2008/0154277 A1
(43) Pub. Date: Jun. 26, 2008

(54) TOOL APPARATUS FOR LOCKING A SPINAL ROD IN AN ANCHORING DEVICE THEREFOR

(76) Inventors: Scott Machalk, Marquette, MI (US); Jeffrey L. Trudeau, Marquette, MI (US); Maria Norman, Negaunee, MI (US); Brian P. Janowski, Marquette, MI (US)

Correspondence Address: FITCH EVEN TABIN AND FLANNERY, 120 SOUTH LA SALLE STREET, SUITE 1600, CHICAGO, IL, 60603-3406, US

(21) Appl. No.: 12/029,846

(22) Filed: Feb. 12, 2008

Related U.S. Application Data
(63) Continuation-in-part of application No. 10/973,659, filed on Oct. 26, 2004.
(60) Provisional application No. 60/889,494, filed on Feb. 12, 2007.

(51) Int. Cl. A61B 17/58; A61B 17/00

(52) U.S. Cl. 606/99; 606/207

(57) ABSTRACT

In one form, a tool apparatus is provided that permits a spinal rod and cap assembly to be secured into a yoke coupling member anchored into a spinal bone. The tool apparatus includes a single actuator handle that is operable to both advance a drive member or persuader rod linearly along a longitudinal axis of the tool and also to rotate the drive member about the longitudinal axis. The rotary motion of the single actuator handle, therefore, is effective to linearly advance a cap assembly and spinal rod into a yoke coupling member secured to the tool and also to rotate at least a portion of the cap assembly to lock the cap relative to the yoke using the same rotary motion of the handle.

### RELATED APPLICATIONS

This application claims the benefit of Application No. 60/889,494, filed Feb. 12, 2006, and is a continuation-in-part of U.S. Utility patent application Ser. No. 10/973,659, filed on Oct. 26, 2004, both of which are hereby incorporated herein by reference in their entirety.

### FIELD OF THE INVENTION

The invention relates to an apparatus for securing a spinal rod along the spine and, more particularly, to a tool apparatus for locking a spinal rod in a coupling member of a spinal rod anchoring device.

### BACKGROUND OF THE INVENTION

In a number of surgical procedures, implant devices are utilized to promote the healing and repair of various parts of the human body. In some cases, implant devices secure bones or bone segments relative to each other so that the bones themselves may heal or fuse. In other cases, implant devices are used to secure a plurality of bones or bone fragments so that soft tissues proximally located to the bones may heal without being disturbed by relative movement of the bones.

Typically, implant devices securing bones or bone segments relative to each other involve securing a plurality of bone screw or other fixtures to a plurality of respective bones. Then, each of the bone screws is secured relative to the others with an additional apparatus, such as a connecting rod. A pedicle screw and rod system is one such example that is commonly used to connect adjacent vertebrae together.

As an example, a patient may require having a number of vertebrae or vertebral fragments secured so that damaged vertebrae may heal and/or fuse. A number of bone screws may be secured to or fastened with a plurality of vertebrae or vertebral segments. Each screw may be integrally attached to or threaded through a coupling member, which often includes opposed, upstanding walls to form a yoke. Each coupling member may be secured with and relative to at least another coupling member with the spinal rod. A locking device is driven into the coupling member to lock the spinal rod relative to the coupling member.

The positioning of the bone screw in a bone is frequently dictated by the size, shape, and surface orientation of the bone. Therefore, when a plurality of bone screws are secured to a plurality of bones or bone fragments, the screws and/or coupling device fixtures are often in a skewed arrangement relative to each other from one vertebra to the next. For this reason, the relative positioning of the bone screws and coupling members can be achieved using the spinal rod to selectively position and orient each bone or bone fragment. Usually, the rod will be bent in a predetermined manner for the desired positioning of vertebrae. However, the deformation provided to the spinal rod prior to its securement with the yokes may not provide exact conformation with the position of the coupling members, thereby requiring force to seat the spinal rods properly within the yokes. For instance, the position of one of the bone screws and coupling members may be shifted by drawing the bone screw and coupling members towards a spinal rod connected to other yokes.

United States Patent Application Publication US 2003/0225408 (“the '408 publication”), to Nichols et al., is directed to an apparatus for securing a spinal rod system with a number of inherent deficiencies. The '408 publication discloses a jaw mechanism for securing jaws to a head portion in which the spinal rod is to be secured, and a rod persuader for advancing the rod toward the head portion. The jaws include a movable jaw and a fixed jaw with the moveable jaw being pivoted by a lever. The lever extends up and away from the body to its proximal end at which a tooth is formed. The lever is biased outward by a leaf spring. To keep the jaws in their closed position against the bias provided by the leaf spring, the toothed end of the lever is received in ratchet teeth on a rack that is pivotally connected to the apparatus and generally extends orthogonally away therefrom.

Accordingly, the lever and rack present a relatively large instrument, which can hinder the ability of a surgeon to operate or see within the surgical site. Furthermore, the outwardly jutting rack and lever are each susceptible to accidental contact, which may result in the rack and lever becoming disengaged. Such disengagement would cause the jaws to release from the yoke. Moreover any force that exists between the rod persuader and the yoke due to the compression being exerted therebetween would be released, which may cause damage to surgical apparatus or to the patient. It has been found in practice that the commercial Nichols et al. tool is not easily disassembled for cleaning and sterilization.

In addition, the tool of the '408 publication requires the surgeon to manipulate two separate handles in order to linearly advance and also rotate a persuader rod holding a locking cap in order to insert and lock the spinal rod relative to the yoke coupling member. A first handle is turned to linearly advance the persuader rod to drive a locking or securing device and spinal rod into the yoke, and a second, separate handle is turned to rotate the persuader rod in order to turn at least a portion of the lock device in the yoke for locking the spinal rod therein. The use of two handles complicates the tool including its operation requiring the surgeon to operate both handles during the operation in order to advance and lock the lock device and spinal rod in the yoke. The second handle can also obstruct the surgeon's view into the surgical site depending on the positions of the two handles.

For this reason, it is desirable to have an improved apparatus for use with such implant devices to direct or manipulate, for instance, a rod into a yoke and effect the securing of the rod therein.

A tool apparatus is provided for securing a lock device and a spinal rod in a yoke coupling member anchored in a spinal bone. In one form, the tool apparatus has a single actuator handle that is operable to both advance a drive rod linearly along the longitudinal axis of the tool and also to rotate the drive rod about the longitudinal axis. The rotary motion of the single actuator handle, therefore, is effective to linearly advance a lock device or cap assembly and spinal rod into a yoke coupling member secured to the tool and also to rotate at least a portion of the cap assembly to lock the cap relative to the yoke using the same rotary motion of the single handle. Accordingly, in this form, a second, separate handle is not required.

In order to switch the tool from an advancing configuration, where turning the single actuator handle linearly advances the rod along the longitudinal axis, to a rotary configuration, where turning the same, single actuator handle rotates the drive rod about the longitudinal axis, the tool apparatus includes a coupling device or switching device to switch between the two tool configurations. Preferably, the switching device is operable to automatically switch between the two tool configurations upon continued rotation of the actuator handle.

In the advancing configuration of the switching device, rotation of the single actuator handle first linearly advances the drive member, generally without rotation thereof, along the longitudinal axis in order to advance the cap assembly and spinal rod into the coupling member secured by a clamping head of the tool. In the rotary configuration of the switching device, rotation of the same, single actuator handle rotates the drive member, generally without linear advancement thereof, in order to rotate at least a portion of the cap assembly to lock the spinal rod in the yoke member.

The single handle is advantageous over prior persuader tools because a surgeon does not need to sequentially manipulate multiple handles to first linearly advance the drive rod and then rotate the drive rod, simplifying the process of reducing and locking the rod in the yoke and eliminating the need for the surgeon to determine or guess when the drive rod is fully linearly advanced and ready to be rotated. Prior persuader tools, on the other hand, employed two handles—one to linearly advance the drive member and one to rotate the drive member. Moreover, the single actuator handle tool herein avoids the potential obstruction created by a second handle to provide the surgeon a better view of the operating site.

### BRIEF DESCRIPTION OF THE DRAWINGS

FIG. 1 is a perspective view of a surgical apparatus in accordance with the present invention showing a clamping mechanism including opposed jaw members clamped onto a coupling member of a spinal rod anchoring device;

FIG. 2 is an exploded perspective view of the surgical apparatus of FIG. 1;

FIGS. 3a and 3b are perspective views of the surgical apparatus showing the jaws of the surgical apparatus in an open position with a clamp actuator for the clamping mechanism pivoted away from the main body;

FIGS. 4a and 4b are perspective views showing the clamp actuator pivoted toward the main body and the jaws in a closed position;

FIGS. 5a and 5b are perspective views showing a drive rod advanced along the main body to push the spinal rod into the coupling member;

FIGS. 6a and 6b are perspective views showing the drive rod turned to lock a securing device onto the spinal rod in the coupling member;

FIG. 7 is a partially exploded perspective view of the main body showing a sleeve coupling subassembly, a drive rod subassembly, a drive sleeve, and a tubular body portion of the surgical apparatus of FIG. 1;

FIG. 8 is a cross-sectional view of a lower portion of the drive sleeve and a torquing portion of the drive rod subassembly;

FIG. 9 is an exploded perspective view of the sleeve coupling subassembly and drive sleeve;

FIG. 10 is a cross-sectional view of the drive sleeve and tubular body portion;

FIG. 11 is a cross-sectional view of the sleeve coupling subassembly, drive rod subassembly, sleeve assembly, and body portion;

FIG. 12 is a perspective view of a second form of a surgical apparatus in accordance with the present invention;

FIG. 13 is a exploded fragmentary view of the surgical apparatus of FIG. 12 showing a main body portion, a drive rod, and a sleeve coupling subassembly;

FIG. 14 is a cross-sectional view of the body and the drive rod showing cooperating structure therebetween taken through the line 14-14 of FIG. 12;

FIG. 15 is a cross-sectional view of the body and the drive rod taken through the line 15-15 of FIG. 12;

FIG. 16 is a perspective view of the drive rod showing a surface of a drive rod including substantially flat portions and a reduced portion;

FIG. 17 is a cross-sectional fragmentary view of the drive rod showing a proximal end thereof;

FIG. 18 is a cross-sectional fragmentary view of the drive rod assembly and an inner portion of a handle for rotating the drive rod assembly;

FIG. 19 is a perspective view of a drive member showing a recess for mating with a portion of the drive rod;

FIG. 20 is a side elevational view of the inner portion of FIG. 18 showing recesses formed thereon;

FIG. 21 is a side elevational view of the inner portion of FIG. 19 showing recesses formed thereon;

FIG. 22 is a cross-sectional fragmentary view of the drive rod and a handle for rotating the drive rod showing an outer portion of the sleeve coupling subassembly engaged in a first position with the inner portion;

FIG. 23 is a cross-sectional fragmentary view corresponding to FIG. 22 showing the outer portion engaged in a second position with the inner portion, and engaged with the drive rod;

FIG. 24 is a perspective view of a third form of a surgical apparatus in accordance with the present invention;

FIG. 25 is a side elevational view of the surgical apparatus of FIG. 24 showing the drive rod in phantom;

FIG. 26 is a cross-sectional fragmentary view of a proximal end of the surgical apparatus of FIG. 24 showing a drive rod cooperating with a sleeve coupling subassembly;

FIG. 27 is a perspective view of a fourth form of a surgical apparatus in accordance with the present invention showing an elongate tool body and a single actuator handle;

FIG. 28 is another perspective view of the surgical apparatus of FIG. 27 showing a yoke coupling member secured in a clamping head at a distal end of the elongate tool body;

FIG. 29 is an exploded view of the surgical apparatus of FIG. 27 showing the actuator handle, coupling device, and elongate tool body;

FIG. 30 is an enlarged perspective view of the elongate tubular body showing one of a pair of diametrically opposed, generally L-shaped guide slots at the peripheral end thereof;

FIG. 31 is a perspective view of the follower member of the coupling device having radial tabs for being received in the guide slot of the elongate tubular member;

FIG. 32 is a perspective view of the follower member showing a drive rod having proximal threads with the follower member fixed adjacent thereto;

FIG. 33 is an enlarged perspective view of the surgical apparatus in an advancing configuration showing one of the follower tabs received in an axial portion of an associated L-shaped guide slot;

FIG. 34 is an enlarged perspective view of the surgical apparatus is in a rotary configuration showing the tab received in a circumferential portion of the L-shaped guide slot;

FIG. 35 is an enlarged perspective view of the surgical apparatus showing the follower member switching from the advancing configuration to the rotary configuration;

FIG. 36 is a cross-sectional view of the surgical apparatus of FIG. 27 showing the drive rod in an initial position with a threaded portion thereof spaced from the follower member;

FIG. 37 is another cross-sectional view of the surgical apparatus of FIG. 27 showing the drive rod in an advanced position with a distal end of the threaded portion thereof engaging the follower member;

FIG. 37A is a cross-sectional view showing an alternative arrangement to couple the handle housing to the locking bearing cap via two corresponding mating grooves and a snap ring;

FIG. 38 is an elevational view of the surgical apparatus of FIG. 27 showing the drive rod in fully advanced position;

FIG. 39 is a fragmentary, cross-sectional view of the clamping head and a drive end of the drive rod showing the drive rod fully advanced with the drive end positioned for rotating the locking device in a yoke coupling member secured to the clamping head of the tubular body;

FIG. 40 is a cross-sectional view of the surgical apparatus showing turning of the actuator handle for linearly advancing or retracting the drive rod member;

FIG. 41 is a plan view of the drive end of the drive rod showing radially extending lobes formed about the drive end and friction inserts between adjacent lobes; and

FIG. 42 is a fragmentary, cross-sectional view taken through line 4L-4R of FIG. 41 showing the friction inserts at the drive rod end.

### DETAILED DESCRIPTION OF THE PREFERRED EMBODIMENT

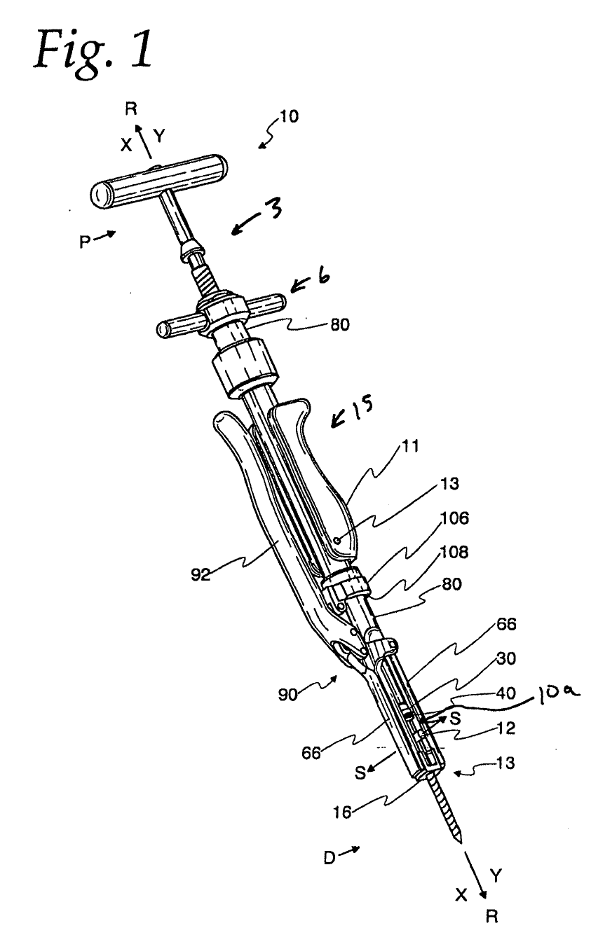

Referring initially to FIG. 1, a rod persuader device 10 for advancing a spinal rod 12 towards a fixation device 14 in the form of a pedicle screw fixture 16 is depicted, the tool 10 having an elongate main body 15 with a distal end D and a proximate end P such that a user would hold and generally operate the persuader 10 toward the proximate end P with the distal end D pointed away from the user.

The main body 15 of the rod persuader tool includes a plurality of elongate members some of which can be shifted longitudinally and/or turned or rotated relative to another member or members(s). The rod persuader tool 10 herein is characterized by its ease of assembly and disassembly to allow for cleaning of its various components on a regular basis. To this end, the tool 10 includes a clamping subassembly 90 including a tubular body portion 80, a drive rod subassembly 3 including a drive rod 140, a rod drive sleeve 132, and a sleeve coupling subassembly 6, which includes a nut 166 threaded to the tubular body portion 80 and a handle sleeve 162 having an internal drive thread 163 to which the drive rod 140 is threaded.

Accordingly, for disassembly, the drive rod 140 is turned to retract it relative to the main body 15 and release the drive rod subassembly 3 therefrom. Thereafter, the nut 166 is turned to retract it along the main body 15 until the sleeve coupling subassembly 6 is released therefrom which, in turn, allows the drive rod sleeve 132 to be pulled out of the tubular body portion 80. This is a fairly quick disassembly procedure that can easily be performed in well less than a minute so that each subassembly 90,3,6, and the sleeve 132 are separated for cleaning. The rod persuader 10 also includes a grip 11 fixed to the body 15 with screw fasteners 13 so that a user may easily manipulate the tool 10, and the grip 11 may easily be separated from the body 15 by loosening the screw fasteners 13.

Similarly, assembly proceeds in an equally easy and quick manner. The tubular body portion 80 may receive the drive sleeve 132 therein, and the sleeve coupling subassembly 6 is threadingly received and advanced on the body portion 80. The drive rod assembly 3 may then be threadingly received and advanced within the sleeve coupling subassembly 6.

The preferred and illustrated rod persuader tool 10 herein is especially well-adapted for use with the spine rod anchoring system described in the commonly assigned co-pending PCT Application No. US04/03605, filed Feb. 5, 2003, the specification of which is incorporated herein by reference in its entirety as if reproduced herein. Generally, the rod persuader tool is used for seating the spinal rod 12 within one or more spinal rod anchoring or fixation devices 14. Preferably, the fixation device 14 includes a screw fixture 16 secured to the pedicle portion of a vertebrae (not shown), such as with a pedicle bone screw 20 extending therefrom. The pedicle screw fixture 16 includes a coupling device, such as a yoke 18 that may be formed unitary with the screw, but preferably the yoke and screw are distinct components for polyaxial anchoring of the screws relative to the coupling member, as described in the PCT US04/03605 Application.

The yoke 18 has a pair of upstanding and opposed walls 22 for receiving the rod therebetween. The spinal rod 12 is captured by a turning of rod securing device 30 including a cam lock member or cap 30a. The preferred securing device 30 includes an intermediate clamping member 30b rotatably secured to the cap 30a by a connector member in the form of a distinct spring clip. To simplify assembly and operation, it is preferred that the tool 10 pushes the spinal rod 12 into the yoke 18 and secures the cap 30a to the yoke 18 to lock at least partially and secure the spinal rod 12 therein. In the preferred embodiment, the cap 30a is set on or removably attached to a gripping or torquing portion 120a of the drive rod subassembly 3 toward the distal end D of the tool 10, the spinal rod 12 may then be set in or otherwise located in a cooperating fashion with a retaining portion 10a of the clamping subassembly 90 toward the distal end D of the tool 10, and the cap 30a and spinal rod 12 are shifted between the walls 22 of the yoke 18.

Once the spinal rod 12 has been urged into and seated in the yoke 18 between its walls 22 so that holding flanges 40 of the cap 30a are aligned with recesses 41 in the yoke walls 22, the cap 30a may then be turned by the drive rod 140 so that the cap 30a is at least partially secured to the yoke 18 with the spinal rod 12 captured therein. To achieve this, the user operates a handle 160 of the sleeve coupling subassembly 6 toward the proximate end P of the tool 10 so that the gripping portion 120a of the drive rod subassembly 3 toward the tool distal end D turns the cap assembly 30 within the yoke 18 for partially locking the spinal rod 12 therein.

During use, the persuader tool 10 is preferably secured to the pedicle screw fixture 16 via the clamping subassembly 90 and, more specifically, opposed clamping jaw members 60,62 thereof. The pedicle screw fixture 16 and the tool 10 are provided with cooperating structure so that the tool 10 may be removably attached to the pedicle screw fixture 16 for operation. The cooperating structure may be one or more recesses that cooperate with one or more projections received therein. As the yoke 18 remains in the patient and is surrounded by living tissue, it is preferred that yoke 18 includes minimal sharp edges, protrusions, or points. Consequently, it is also preferred that the walls 22 of the yoke 18 include the recesses for receiving the corresponding projections on the jaw members 60, 62 of the persuader tool 10.

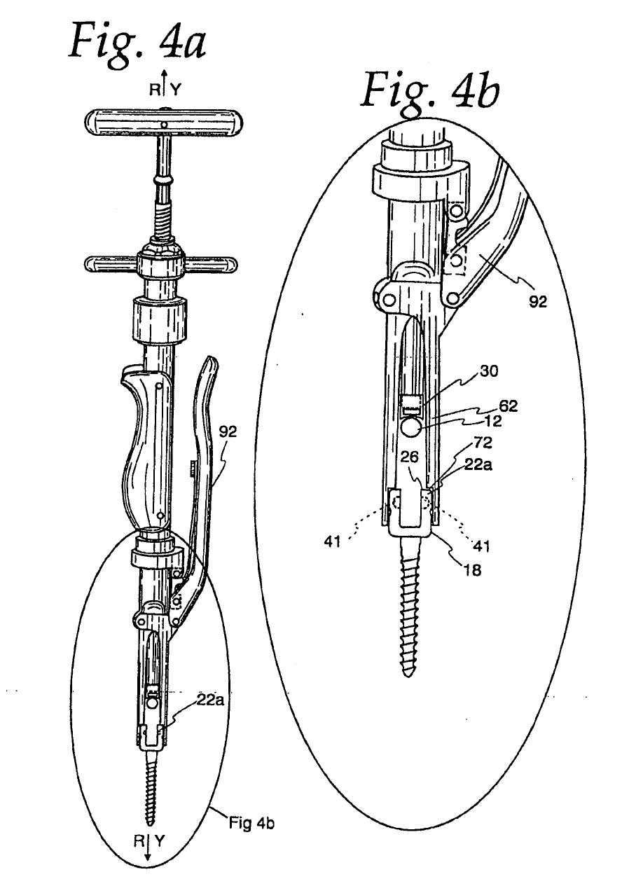

The clamping mechanism 90 herein preferably provides the tool 10 with a relatively compact configuration, particularly with the tool 10 in the clamped state where the yoke 18 is clamped between the jaw members 60,62, as shown in FIGS. 4a-6b. This compact configuration is particularly important during a spinal rod securing procedure since advancing the rod securing assembly 30 via operation of the handle 146 of the drive rod subassembly 3 and turning the cap 30a via operation of the handle 160 of the coupling subassembly 6 all occur with the tool in its clamped, compact configuration. In this regard, the tool clamping subassembly 90 has a clamp actuator or lever 92 than pivots one of the jaw members, particularly movable jaw 62, with the lever 92 being pivotally connected to a relatively small link member 100 that extends between the lever 92 and the tool body 15 to provide the clamping force exerted by the jaw members 60,62 on the yoke 18, as will be more fully described hereinafter.

As shown, in the clamped state, the lever 92 is pivoted toward the tool body 15 so that it generally extends along the axis R thereof. By way of example, in practice the tool 10 can be effectively implemented so that the lever 92 is within approximately one inch or less of the tool axis R at any point therealong. Moreover, unlike the rack in the previously-discussed Nichols, et al., tool there are no tool components used for the clamping operation of the present tool 10 that extend substantially transverse or orthogonal to the tool axis R, and in any event well beyond the lever 92.

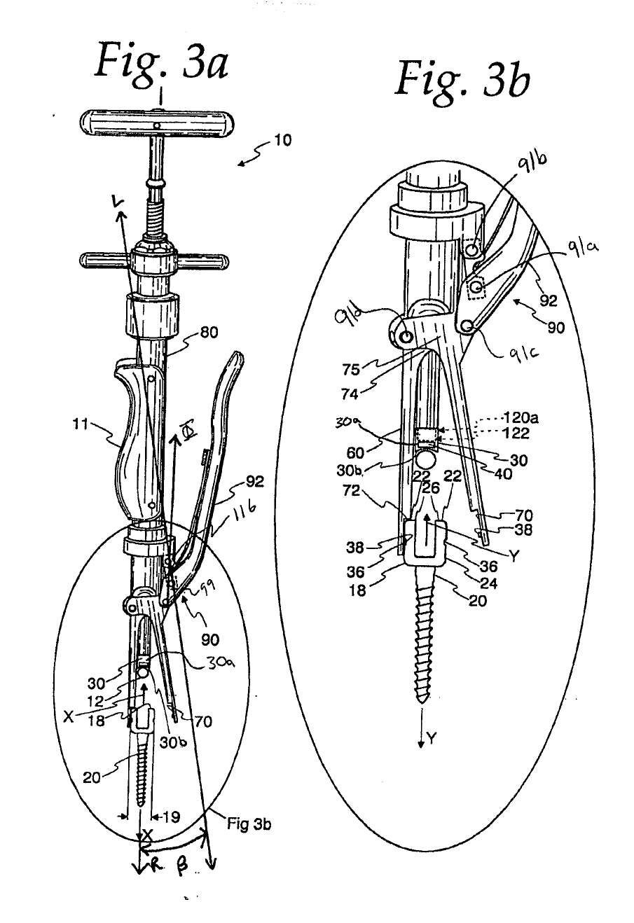

Even in the open or unclamped state, the actuating lever 92 does not significantly increase the effective width of the tool body 15 in the direction transverse to the tool body longitudinal axis R (FIG. I), as shown in FIG. 3a. For example in practice the tool 10 can be effectively implemented so that with the lever 92 pivoted open, the free end 92a of the lever is spaced by approximately four inches or less from the tool axis R. Further, again unlike the rack of the previously discussed Nichols, et al., tool, there are no other tool components that extend beyond the lever end 92a spaced from the tool axis R.

Continuing reference to FIG. 3a, the yoke 18 has a central longitudinal axis Y which may or may not be aligned with a central longitudinal axis X of the pedicle bone screw 20 secured to a vertebra. The spinal rod 12 also has a central longitudinal axis S which, when seated in the yoke 18, is transverse and, ideally, orthogonal to the axis Y of the yoke 18.

More particularly, it is preferred that the tool 10 directs the cap 30 and the spinal rod 12 along the path defined by the axis Y of the yoke 18. To achieve this, it is further preferred that the cooperating recesses and protrusions of the yoke and tool 10, respectively, provide a generally pre-determined orientation when the tool 10 is attached to the yoke 18. In the present embodiment, the recesses are generally oval-shaped recesses 36, and each projection is a generally oval-shaped tooth 38 that mates with a recess 36 in a specific relative orientation. In this manner, the attached tool 10 directs the movement of the cap 30 and spinal rod 12 along the axis Y of the yoke 18.

The yoke 18 is generally rigidly formed, and the recesses 36 are preferably located on an outer surface 50 of the yoke 18. In the present embodiment, the yoke 18 includes two recesses 36, one in each wall 22, such that the recesses 36 are outwardly opposed from each other and lie in a line perpendicular or orthogonal to both the axis Y of the yoke 18 and the axis S of the spinal rod 12 when seated. The configuration of the paired recesses 36 and teeth 38 provides balanced transmission of the force from the tool 10 directing the spinal rod 12 into the yoke 18 through the mating recesses 36 and teeth 38.

As previously discussed, the tool 10 includes a pair of opposed jaws 60 and 62, and each jaw 60,62 includes a tooth 38. One of the jaws 60,62 is movable relative to the tubular body portion 80 such that the jaws 60,62 may open and close relative to each other for attaching or releasing from the pedicle screw fixture 16. More specifically, in the orthogonal direction to the tool axis R, the jaws 60,62 may be opened so the teeth projections 38 are spaced by a distance greater than an outer dimension 19 of the yoke 18 (see FIG. 3a). In this position, the teeth 38 of the jaws 60, 62 have clearance for being positioned around the yoke 18 or for being removed from the yoke 18. To attach the jaws 60,62 and tool 10 to the yoke 18, the jaws 60,62 are moved together, or closed, so that the teeth 38 on each jaw 60,62 are received in one of the recesses 36 on the walls 22 of the yoke 18.

Each jaw 60,62 includes a terminal portion 64 and a jaw body 66 having a securement end 68. The terminal end 64 includes the tooth 38 and is clamped to the yoke 18 during the spinal rod anchoring operation with the tool operative to push the cap 30 and rod 12 into the yoke 18 and turn the cap 30 for partial locking of the cap 30 and spinal rod 12 relative to the yoke 18. The walls 22 of the yoke 18 preferably have a generally cylindrical exterior surface 24 in which the recesses 36 are formed. Accordingly, the terminal end 64 has an interior surface 70 surrounding the tooth 38 that is arcuate to conform generally to the exterior surface 24 of the walls 22. The jaw body 66 includes a transverse shoulder 72 from which the terminal end portion 64 depends, and the shoulder 72 is shaped and positioned such that it substantially abuts flush against a top surface 26 of the yoke walls 22 (see FIG. 3b). The generally matching contours of the wall exterior surface 24 and the terminal end interior surface 70, as well as the shoulder 72 abutment with the yoke wall top surface 26, thereby assist in constraining the jaws 60,62 and yoke 18 to a specific relative orientation during operation, as described above.

In the preferred and illustrated embodiment, jaw 60 is stationary and jaw 62 is movable relative to the tubular body portion 80 of the tool 10, as previously mentioned. More specifically, stationary jaw 60 is formed integral with or fixedly attached to the tubular body portion 80 so as to form a generally unitary structure. Movable jaw 62 is pivotally secured with the clamping subassembly 90 including the movable jaw 62 to the tubular body portion 80 and to the stationary jaw 60.

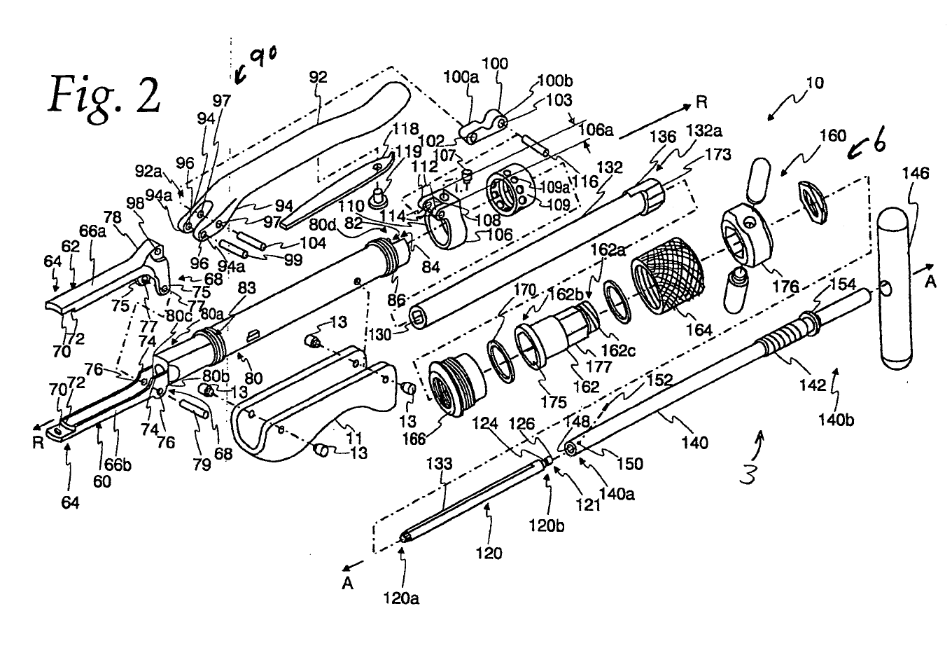

As can be seen in FIG. 2, the jaw body 66a of the stationary jaw 60 is secured by its securement end 68 to a distal end 80a and side edge 80b of the tubular body portion 80. The stationary jaw body 66a includes spaced sidewall portions 74 which extend to the body portion distal end 80a from the side edge 80b towards an opposite side edge 80c of the distal end 80a. The sidewalls 74 provide additional support for the stationary jaw 60 to be joined with the tubular body portion 80.

The movable jaw body 66a is secured so that it is generally positioned at the side edge 80c of the tubular body portion 80. The jaw body 66b of the movable jaw 62 includes sidewalls 75 extending generally in an inward direction towards the stationary jaw 60, and includes a pivot block 78 extending in an opposite, generally outward direction.

The jaw sidewalls 74,75 each include respective transverse apertures 76,77 aligned with the transverse aperture 76,77 of the other sidewall 74,75 such that the apertures 76 and 77 are aligned generally orthogonally to a central longitudinal axis R of the rod tool 10. In addition, the bores 76 of the sidewall 74 are also aligned with the bores 77 of the sidewall 75 such that a pivot pin 79 may be secured therein permitting the movable jaw 62 and stationary jaw 60 to pivot relative to each other around the pivot pin 79 and bores 76,77.

The pivot block 78 of the movable jaw 62 is connected to the clamping mechanism 90. The clamping mechanism 90 includes the lever 92 that has a distal end 92a including a pair of arms 94. The arms or tines 94 include first and second pairs of transverse bores 96,97 where the bores of each pair aligned are with each other and aligned generally orthogonally to the axis R of the tool 10. At least terminal portions 94a of the arms 94 are separated by a distance sufficient to allow the pivot block 78 of the movable jaw 62 to be received therebetween. When positioned in this manner, the first bores 96 are aligned with a pivot bore 98 in the pivot block 78 that is also transverse and generally orthogonally oriented relative to the axis R of the tool 10. A pivot pin 99 may be secured within the first bores 96 of the lever 92 and the pivot bore 98 such that the movable jaw 62 and the lever 92 may pivot relative to each other about the pivot pin 99 and the bores 96, 98.

The link 100 of the clamping mechanism 90 is provided and is pivotally attached to both the tool body 15 and the lever actuator 92, as previously discussed. The link 100 is sized relative to pivotal connections so that it assists in generating and transmitting the clamping force at the jaw members 60,62 on the yoke 18 when the lever 92 is pivoted toward the tool body 15 to its clamped position, as will be described more fully hereinafter. More specifically, the link 100 has first and second bores 102,103 where each bore is aligned generally orthogonally to the axis R of the tool 10. The first bore 102 is located proximate to a displaceable end 100a of the link 100, and the displaceable end 100a is sized to be received between the arms 94 of the lever 92. As such, the first bore 102 of the link 100 may be aligned with the second bores 97 of the lever 92, and a pin 104 may be secured therein to allow the lever 92 and link 100 to pivot relative to each other about the pin 104 received in the bores 102, 97.

To pivotally connect the link 100 to the tool body 15, a collar 106 is provided. The collar 106 has a ring-like structure secured around the tubular body portion 80 and fixedly attached thereto, such as by set screw 107. In the preferred embodiment, an adjustment device 109 is provided with the collar 106 which allows the clamping force generated by the clamping mechanism of the tool 10 to be adjusted. More specifically, the tubular body portion 80 includes a threaded portion 83 onto which the adjustment device in the form of annular adjustment ring 109 is threadably received. The adjustment ring 109 may be adjustably positioned on the tubular body portion 80 by rotating the adjustment ring 109 along the threads 83. The collar 106 is sized to extend around the adjustment ring 109 so that when the set screw 107 is disengaged from the adjustment ring 109, the adjustment ring 109 may rotate relative to the collar 106 to shift along the longitudinal axis R of the tool 10. Once the adjustment ring 109 is in a desired position, the set screw 107 is advanced to secure the collar 106 to the adjustment ring 109, which may include surface features such as dimples or recesses 109a for receiving the advanced set screw 107.

The collar 106 has a greater outer dimension 106a than the tubular body portion 80 (see FIG. 2) such that the collar 106 forms a shoulder 108 with and extending generally radially from the tubular body portion 80. The shoulder 108 includes a surface 110 at least partially directed toward the distal end D of the rod tool 10. The surface 110 includes a pair of link mount portions or fingers 112 generally extending from the surface 110 and towards the distal end D. Preferably, the surface 108 lies in a plane perpendicular to the longitudinal axis R of the tool 10, and the fingers 112 extend orthogonally from the surface 108 towards the distal end D such that the fingers 112 extend generally in a direction parallel to the longitudinal axis R of the tool 10.

Each finger 112 includes a bore 114 where each bore is aligned with the other and aligned generally orthogonally to the axis R of the tool 10. The fingers 112 are relatively positioned such that a pivotal end 100b of the link 100 may fit therebetween. The link 100 is pivotally secured to the fingers 112 of the collar 106 by a pin 116 received in the apertures 114.

In operation, a user operates the lever 92 between a clamped position (see FIGS. 4a, 4b) and an unclamped position (see FIGS. 3a, 3b) to open and close the jaws 60,62 on the yoke 18. More specifically, the lever 92 is moved to the open position by pulling outward on the lever 92. As such, the pivot point or connection defined by the second bores 97 of the arms 94 of the lever 92 is displaced outwardly. The second arm bores 97 are connected to the displaceable end 100a of the link 100 such that the displaceable end 100a is shifted outwardly. However, the link 100 is connected also to the collar 106 by the pivotal end 100a such that the link pivotal end 100a pivots relative to the fixed collar 106. Consequently, as the second arm bores 97 of the lever 92 are displaced outwardly, they also shift in a rearward direction toward the proximal end of the tool 10.

Accordingly, the first arm bores 96 of the lever 92 are displaced inwardly, as well as rearwardly. As the first arm bores 96 are pivotally connected to the pivot block 78 of the movable jaw 62, the pivot block bore 98 is displaced inwardly and rearwardly. This allows the movable jaw 62 to rotate or pivot around its bores 77 such that the jaw 62 is moved to the open position.

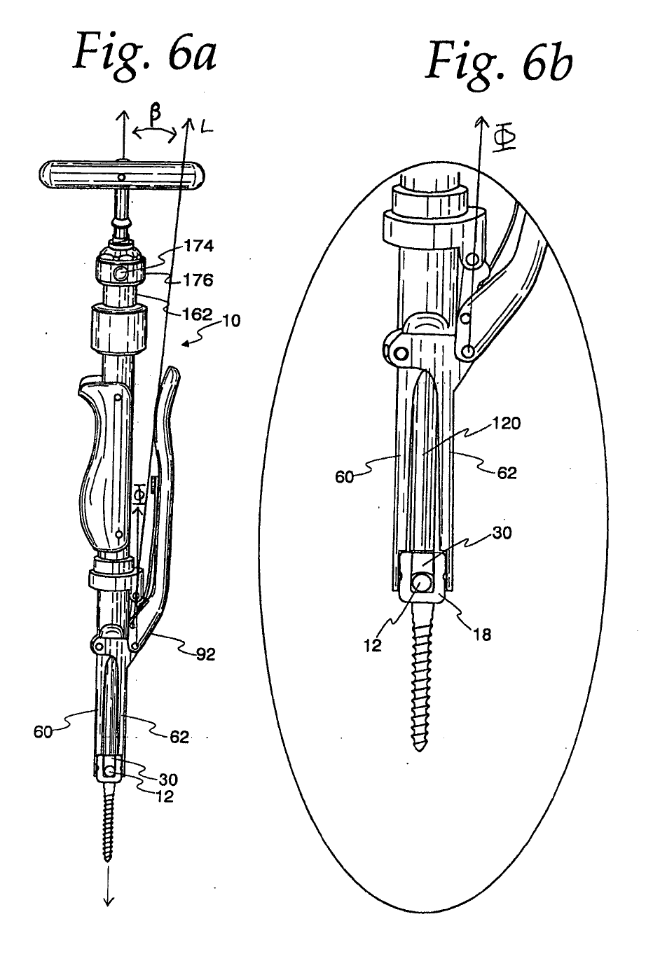

To close the jaws 60,62, the lever 92 is displaced inwardly toward the tubular body portion 80 of the tool 10. The link 100 has a longitudinal plane L defined by the axes of the block line bores 102,103. When the lever 92 is in an open position such that the jaws 60,62 are in an open position, the plane L of the link 100 and the persuader axis R form a positive oblique angle P (see FIG. 3a). As the lever 92 is displaced inwardly toward the tubular body portion 80 to close the jaws 60,62, the link 100 pivots such that the angle P between the link plane L and the axis R decreases. The angle decreases to 0°, at which point the link plane L and persuader axis R are parallel. In order to fully close, the link 100 is rotated an additional amount such that the angle P is negative and the link plane L and persuader axis R are oblique (see FIG. 6a).

More specifically, when the lever 92 shifts from the open position to the closed position, the pin 104 connecting the lever 92 and link 100 passes a line extending between the pins 99 and 116 connecting the lever 92 to the movable jaw 62 and connecting the link 100 to the collar 106, respectively. Accordingly, the distance between the pins 99 and 116 varies as the clamping assembly 90 is pivoted. The point and moment at which the distance between the pins 99 and 116 is greatest is as the pin 104 passes between the pins 99 and 116.

Prior to this moment, the jaws 60,62 are sized and arranged relative to each other and the size of the yoke 18 such that they are in flush contact with the yoke 18. When in this flush contact, the terminal portions 64 of the jaws 60,62 are substantially unable to shift closer together to allow the clamping assembly 90 to pivot towards the closed position without the application of increased force.

Upon application of an increased force to the lever 92, the components of the clamping mechanism 90 such as at pivot connections 91a-91d, the link 100 and jaws 60,62 may flex or deform a small amount to permit the pin 104 to pass between the pins 99 and 116. In addition, any play between the clamping mechanism components such as provided by manufacturing tolerances will be taken up during such higher force clamp actuator lever pivoting. Once the pin 104 has passed between the line formed between the pins 99 and 116, the distance required between the pins 99 and 116 for the pins 99,104, and 116 to be aligned is no longer necessary and, hence, the distance between the pins 99 and 116 decreases with the continued operation the clamping assembly 90. Accordingly, the jaws 60, 62 and linkages decrease the amount they are flexed as the force is relieved. In order to release the jaws 60, 62, the lever 92 is shifted from the closed position towards the open position, and, to do so, the pin 104 must again pass between the pins 99 and 116. Thus, an application of force must be exerted to impart again the flex to the pivot connections and jaws 60,62 and link 100 to shift the pin 104 between the pins 99 and 116. In this manner, the clamping assembly 90, including the lever 92, and the movable jaw 62 form a vise-grip type compression-lock such that the jaws 60,62 are clamped without requiring a user to maintain clamping pressure and such that a user may release the clamping by pivoting the lever 92 outwardly.

As described above and with reference to FIG. 3b, the clamping mechanism 90 includes pivot connections 91a-91d with the arrangement of these pivot connections generating the clamping force applied by the jaw members on the yoke 18 to form a compression lock therebetween. While the pivot connection 91a between the link 100 and clamp actuating lever 92 and the pivot connection 91d between the jaws are substantially fixed, it is the movement of the other pivot connections 91b, 91c relative to pivot connection 91a that dictates the clamping force generated by the clamping mechanism 90. In this regard, shifting the adjusting device 109 along the tool body 15 changes the location of the pivot connection 91a for adjusting the applied clamping force on the yoke 18, as previously described.

The jaw pivot connection 91d is operable to allow the pivot connection 91c between the lever 92 and movable jaw member 62 to be displaced as the lever 92 pivots. The pivot connection 91b between the lever 92 and link 100 is spaced further from the tool axis R than the pivot connections 91a and 91c when the tool 10 is in its unclamped state. With the yoke 18 in place for clamping, pivoting the lever 92 causes the jaw members 60,62 to tightly engage the yoke 18 with the pivot connection 91b shifting toward the tool axis R, and the pivot connection 91c shifting down along the tool axis R toward tool distal end D. Once tightly engaged in the yoke 18, continued lever pivoting causes increased clamping pressure on the yoke 18 as the pivot connections 91b and 91c continue to shift as described above. This increased clamping pressure generates reactive forces so that any play in the pivot connections 91a-d is taken up, along with some very slight and non-damaging deformation of the generally rigid components of the clamping mechanism 90, such as the elongate jaw members 60,62 and/or lever 92.

With the pivot connection 91b shifted into alignment with pivot connections 91a and 91c, the pivot connection 91c is shifted down the tool axis R to its maximum point of separation from the fixed pivot connection 91a so that maximum clamping force is generated by the jaws 60,62 with the pivot connections 91a-c in this straight-line orientation.

Eventually, the pivot connection 91b is shifted sufficiently to where it passes the straight line formed between pivot connections 91a and 91c on either side thereof and moves closer to the tool axis R. Generally, at this point there already is more than sufficient clamping pressure so that this continued pivoting of the clamp lever 92 simply serves to alleviate the stress in the clamping mechanism 90 components. In fact, this shifting to the over-the-line position of the pivot connection 91b is accompanied by a reduction in the user-applied force necessary for such continued lever shifting as the stress induced in the clamping mechanism 90 components is greatest in the straight-line arrangement of the pivot connections 91a-c so that it becomes easier to push the lever 92 to shift the pivot connections out of alignment with pivot connections 91a and 91c. This arrangement also provides for secure clamping since once the pivot connection 91b has been shifted passed the line between pivot connections 91a and 91c, the clamping mechanism 90 is substantially locked or retained in this clamping position of the pivot connections 91a-c because of the high force that must be applied to shift the pivot connection 91b back passed the line to overcome the high stresses induced in the clamping mechanism 90 components. Accordingly, for this purpose, it is necessary for the user to pull on the free end of the clamp lever 92 which provides a lever arm advantage in permitting the user to shift the pivot connection 91b back to the other side of the line spaced further from the tool axis R than pivot connections 91a and 91c. A spring assist can also be provided to urge the lever 92 to its pivoted open configuration, as will be described hereinafter. In this manner, the present clamping mechanism 90 is also relatively compact in the way it integrates a clamp force retention mechanism with the clamp force generating function via the pivot connections 91a-c that are oriented along only a small section of the tool body 15, e.g., approximately one inch or less in practice, with pivot connection 91b only spaced by approximately three quarters of an inch or less in practice, from the tool axis R in the unclamped state of the tool 10.

As previously mentioned, changing the position of the adjustment ring 109 permits the clamping force provided by the above-described compression-lock to be adjusted. More specifically, the position of the collar 106 may be adjusted by disengaging the set screw 107 from the adjustment ring 109, and rotationally shifting the adjustment ring 109 along the threads 83 of the tubular body portion 80. As this is done, the distance between the pins 99 and 116 is adjusted. As this distance decreases, a greater force must be applied to the lever 92 to drive the pivot connections 91a and 91c a sufficient distance apart so that pivot connection 91b can be shifted as described earlier. In other words, with a smaller distance between the pivot connections 91a and 91c, it becomes increasingly difficult to force the link 100 and jaws 60,62 to pivot so the pin 104 may pass between the pins 99 and 116. Conversely, as this distance increases, a lower force is required to effect this pivoting.

A leaf spring 118 may be provided that is secured by a set screw or rivet 119 to the lever 92 at a fixed end thereof to urge the lever 92 toward its open position to assist the user in opening the jaws, as previously described. In addition, the leaf spring 118 assists in holding the lever 92 outwardly when in the open position.

The leaf spring 118 includes a free end 118a that engages and rides on a facing or upper surface 100a of the pivoting link 100. When the tool 10 is in the open position, the leaf spring 118 biases the clamp lever 92 and link 100 to pivot open about the pivot connection 91b. In this manner, the bias force provided by the leaf spring 118 acts in conjunction with the stress forces in the clamping mechanism 90 that tend to drive and keep the pivot connection 91b spaced further from the tool axis R than the pivot connections 91a and 91c. It is only when the pivot connection 91b is shifted to be in alignment with pivot connections 91a and 91c that the clamping mechanism 90 is in sort of a temporary neutral state with pivot connection 91b neither urged toward or away from the tool axis R by the stresses induced in the clamping mechanism 90 by pivoting of the clamp lever 92. When in the closed position, the bias force provided by the leaf spring 118 between the link 100 and the lever 92 is well below that required to overcome the stress forces in the clamping mechanism 90 that tends to drive and keep the pivot connection 91b closer to the tool axis R than the pivot connections 91a and 91c.

Prior to attaching the tool 10 to the yoke 18, the spinal rod 12 is typically placed between the jaws 60,62. It should be noted that a free, completely unsecured spinal rod 12 may be inserted laterally between the jaws 60,62 after the jaws 60,62 have been attached to the yoke 18. However, in such a case, it is unlikely that the tool 10 would be necessary to force or direct a free spinal rod 12 into the yoke 18. Accordingly, it is preferred that the spinal rod 12 is initially located between the jaws 60,62. In the preferred embodiment, the cap 30a is utilized for capturing and/or securing the spinal rod 12 within the yoke, and the cap 30a is located or positioned between the jaws 60, 62 prior to insertion of the spinal rod 12 between the jaws 60,62 so that a drive end 120a of the tool shaft 120 is received in a recess 122 in the cap 30a (see FIGS. 3a, 3b).

Once the jaws 60,62 are secured to the yoke 18 as described for directing the cap 30a and spinal rod 12 into the yoke 18, the tool 10 generally remains stationary as the cap 30a and spinal rod 12 are forced towards and into the yoke 18. The tool 10 includes a movable member of the drive rod assembly 3, the movement of which effects the shifting of the cap 30a and spinal rod 12. In the preferred embodiment, the movable member is a tool shaft 120 which translates linearly along the tool axis R and axis Y of the clamped yoke 18. In the present form, the jaws 60, 62 are connected to the tubular body portion 80 of the tool 10, and the tool shaft 120 is retained by the tubular body portion 80 to permit translation of the tool shaft 120 relative to the tubular body portion 80.

Preferably, the tool shaft 120 is an elongated rod-like member received within an elongate, longitudinal throughbore 82 of the tubular body portion 80. The tool shaft 120 advances along the axis R of the persuader to push the cap 30a and spinal rod 12 into the yoke 18. More specifically, the tool shaft 120 engages and pushes against the cap 30a, which in turn causes the saddle 30b to contact and advance against the spinal rod 12 such that the rod securing device 30 and spinal rod 12 are advanced into the yoke 18.

As the spinal rod 12 is advanced toward and into the yoke 18, the spinal rod 12 is unable to rotate due to the tight clamping of the yoke walls 22 with the jaws 60,62. The saddle 30b in contact with the spinal rod 12 preferably also does not rotate during advancement. The cap 30a may be provided with a structure that may only be advanced between the yoke walls 22 in a particular orientation that does not provide for rotation. For instance, the cap 30a may have a central cylindrical body sized for being received closely and rotating within the yoke walls 22, yet also having lateral holding flanges 40 that extend outward through spaces between the walls 22 such that rotation is prevented unless the holding flanges 40 are advanced into the yoke 18 such that they are aligned with recesses 41 formed in the walls 22. At such a point, the holding flanges 40 may rotate into the recesses 41 to secure the cap 30a to the yoke 18. In addition, the cap 30a and saddle 30b do not rotate against the spinal rod 12 to minimize friction and damage between the securing device 30 and spinal rod 12 during advancement.

Though it is preferred that the cap 30a does not rotate during advancement, the cap 30a is preferably rotated to lock or at least partially capture the cap 30 within the yoke walls 22 once the spinal rod 12 is seated. In order to provide for this securing rotation, the cap 30a includes the drive recess 122 in which a mating drive end portion 120a of the tool shaft 120 is received. So that the cap 30a is not rotated until it is being secured, the tool shaft 120 is also restricted from rotating until such cap rotation is undertaken. Therefore, during advancement of the cap 30a and spinal rod 12, the tool shaft 120 advances linearly and non-rotationally. It should be noted that the securing rotation may be partial such that the cap 30a and spinal rod 12 are not fully locked, in which case the cap 30a is partially secured in the yoke 18. Alternatively, the cap may be turned to be fully locked in the yoke 18 with consequent full locking and seating of the spinal rod 12.

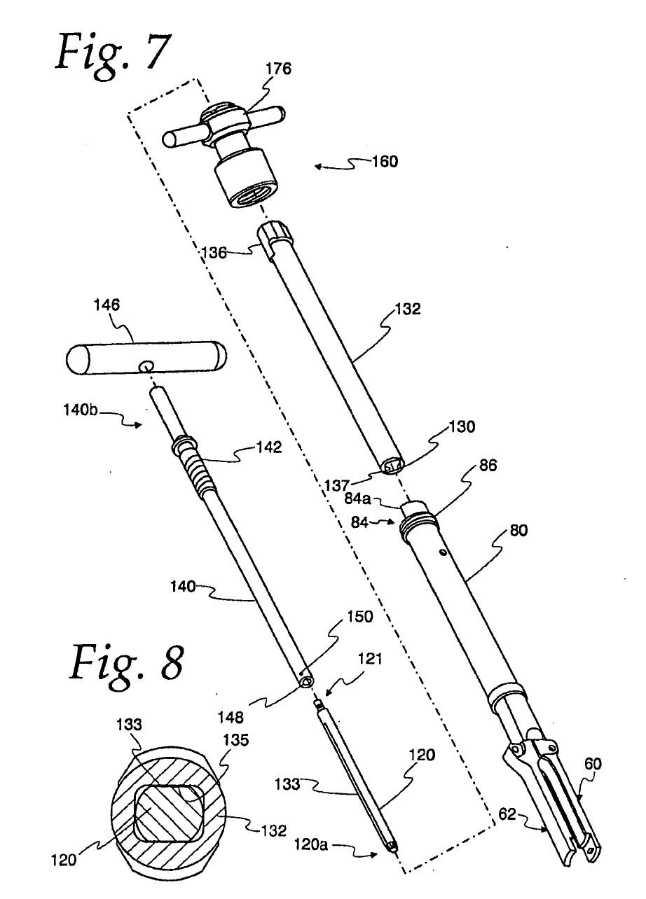

A number of systems may be utilized for advancing the tool shaft 120 and cap 30a linearly, as described, until the cap 30a is aligned with recesses 41 in the yoke 18, whereupon the cap 30a and tool shaft 120 are rotated to capture at least partially the cap 30 within the yoke 18. Referring specifically to the embodiment depicted in FIGS. 1-10, the tool shaft 120 is received within a longitudinal throughbore 130 in the drive sleeve 132, which is in turn received in the throughbore 82 of the tubular body portion 80.

The tool shaft 120 and drive sleeve 132 have cooperating structure such that the tool shaft 120 and drive sleeve 132 are generally prevented from rotating relative to each other, while permitting the linear translation of the tool shaft 120 within the drive sleeve 132. In the preferred embodiment, the tool shaft 120 has an other than circular cross-sectional shape, as does the drive sleeve 132. The tool shaft 120 and drive sleeve 132 may have substantially similar cross-sectional shapes, though it is preferred that they are dissimilar so as to reduce surface contact, thus reducing friction therebetween. For instance, the tool shaft 120 may be in the shape of a cylinder that has truncated sides along parallel cord lines in order to produce flat surfaces 133 on opposite sides of the tool shaft 120, as can be seen in FIGS. 2 and 10. Also referring to FIG. 10, the drive sleeve 132 is depicted with the longitudinal throughbore 130 therein having a rectangular geometry in order to produce flat surfaces 137 that mate and slidingly abut the flat surfaces 133 on the tool shaft 120.

In order to advance the tool shaft 120 relative to the drive sleeve 132, a is provided, at least a portion of which is received within the drive sleeve 132. The drive rod 140 and tool shaft 120 are cooperatively connected such that the tool shaft 120 and drive rod 140 move together along the axis A of the drive rod 140 in a generally linear manner. Accordingly, the tool shaft 120 has a connecting end 120b opposite from the drive end 120a that is joined to a connecting end 140a of the drive rod 140.

The drive rod 140 is threadably advanced or retracted in order to control its movement. More specifically, the drive rod 140 includes an externally threaded portion 142 that mates with an internally threaded portion 134 of the drive sleeve 132. The drive rod 140 has a operable end 140b to which a T-shaped drive handle 146 is connected. In this manner, torque generated by a user in rotating the drive rod 140 via the drive handle 146 advances the drive rod 140 along the threads so that the force generated between the tool shaft 120 and the spinal rod 12 being advanced does not cause the drive rod 140 to be reverse rotated.

The drive rod 140 is connected to the tool shaft 120 so that it may threadably rotate independent of and advance without rotating the tool shaft 120 and the drive sleeve 132. The tool shaft connecting end 120b for connecting to the drive rod 140 has a reduced integral post portion 121 received within a cylindrical recess 148 in the driving rod connecting end 140a. The drive rod 140 has a pair of bores 150 extending therethrough and through the recess 148. The bores 150 are offset from a diametral line so that they are aligned with an annular groove 124 of the post portion 121. A pin 152 inserted through the bores 150 passes through the annular groove 124 so the post portion 121 is captured within the recess 148, and linear movement in either direction by the drive rod 140 causes generally identical linear movement by the tool shaft 120. However, as the post portion 121 is free to rotate relative to the pin 152, the tool shaft 120 generally does not rotate due the rotational movement of the drive rod 140.

Accordingly, the drive handle 146 is rotated such that the mating threads 134,142 of the drive sleeve 132 and drive rod 140, respectively, effect linear movement of the drive rod 140 along its longitudinal axis A. Though connected to the drive rod 140, the tool shaft 120 does not rotate, instead only moving linearly. When this linear movement by the tool shaft 120 is towards the distal end D of the tool 10, the movement is transmitted to the cap 30 and spinal rod 12 to force the cap 30 and spinal rod 12 into the yoke walls 22.

Once the cap 30a and spinal rod 12 are between the yoke walls 22, it is desirable to secure or capture at least partially the cap 30a therein. The advancement of the cap 30 and spinal rod 12 will generally cease when the spinal rod 12 is seated. Preferably, at such a point, the laterally extending holding flanges 40 on the cap 30 are aligned with the yoke recesses 41 for receiving the holding flanges 40 therein. At this point, the drive sleeve 132, tool shaft 120, and drive rod 140 may be rotated together to rotate the holding flanges 40 into the recesses 41 to capture the cap 30 at least partially within the yoke 18.

Preferably, the drive rod 140 is equipped with a structure defining the maximum amount of relative rotation between the drive rod 140 and the drive sleeve 132. The tool 10 is designed so that threadably rotating the drive rod 140 into the shaft sleeve a predetermined amount results in the holding flanges 40 of cap 30a being aligned with recesses 41 in the yoke 18.

Toward this end in the preferred and illustrated form, the drive rod 140 is provided with a radially extending annular shoulder 154 intermediate the threaded portion 142 and the drive handle 146. The drive rod 140 may be advanced until the driving rod shoulder 154 abuts a shoulder 156 located on the sleeve coupling subassembly 6, and the sleeve coupling subassembly 6 is rotated to rotate the drive sleeve 132, tool shaft 120, and drive rod 140 together to capture at least partially the cap 30a within the yoke 18.

The sleeve coupling subassembly 6 includes a securing handle 160 operatively connected to the drive sleeve 132. Once the driving rod shoulder 154 contacts the shoulder 156, the driving handle 146 is no longer rotated. If rotation is continued beyond a certain point, the drive rod 140 can become tightly bound at its threads and/or the shoulders 154,156. During removal, such binding may cause the cap 30a to be loosened or released.

More specifically, if the tool shaft 120 is counter-rotated (rotates opposite the direction of rotation for advancing the spinal rod 12 into the yoke 18) during extraction of the drive end 120a from the cap recess 122, the cap 30a may also be counter-rotated to a loosened or released position from the yoke 18. Therefore, it is preferred in this extraction that the tool shaft 120 linearly retracts.

This extraction of the tool shaft 120 is effected by counter-rotating the drive rod 140. If the drive rod 140 were bound by its threads and/or the shoulders 154,156, counter-rotation of the drive rod 140 would also counter-rotate the drive sleeve 132, which in turn would cause counter-rotation of the tool shaft 120.

Accordingly, the drive sleeve 132 is operatively connected to the securing handle 160 and, when the shoulders 154,156 of the drive rod assembly 3 and sleeve coupling assembly 6 contact, the securing handle 160 is rotated to at least partially lock the cap 30a and spinal rod 12. In this manner, rotation of the securing handle 160 rotates the cap 30a without advancing the drive rod 140. As the cap 30a approaches being fully seated, the drive sleeve 132 will have a tendency to rotate a small amount from the increased force and friction. When this happens, a user may recognize the rotation because the securing handle 160 may also rotate. It is preferably at this point, which coincides with the shoulders 154,156 contacting, that the user then rotates the securing handle 160 to at least partially secure the cap 30a.

In the preferred embodiment, the cap 30a does not rotate, or rotates a limited amount as it is being advanced into the yoke 18, until being fully advanced within the yoke 18. To secure the advanced cap 30a, the drive sleeve 132 is rotated by rotating the securing handle 160 relative to the tubular body portion 80.

Due to a limited ability to view the surgical site, it is difficult, if not impossible, for a user to recognize the degree to which the cap 30 has been rotated relative to the yoke 18. For this reason, it is preferable to provide the user with a tactile indication of complete rotation. In the present embodiment, this tactile indication is provided by cooperating structure between the drive sleeve 132 and the tubular body portion 80, as best viewed in FIG. 10. The tubular body portion 80 is generally cylindrical and includes a circumferentially extending notch or slot 84 that is open to its proximal end 80d, and the drive sleeve 132 is generally cylindrical and includes an integral boss 136 raised from the outer circumferential surface 132b of the sleeve 132 toward the proximal portion 132a thereof. The integral boss 136 is narrower than the circumferential slot opening 84 in that it does not extend as far as the slot 84 in the circumferential direction. When the sleeve 132 is slid into the bore 82 of the tubular body portion 80, the boss 136 fits between the angularly or circumferentially spaced edges 84a and 84b of the slot opening 84. Accordingly, the relative sizes of the slot opening 84 and boss 136 are selected to provide a desired amount of rotary motion for the cap 30a from its unlocked position relative to the spine rod to either a predetermined partially locked position or fully locked position. In use, the securing handle 160 is initially positioned so that the shaft sleeve boss 136 is against a first edge 84a of the notch 84. The cap 30a is placed on the tool shaft drive end 120a such that the cap holding flanges 40 are positioned in gaps 61 between the jaws 60,62 so that the jaws 60,62 may be secured to the yoke 18 for advancing the cap 30a.

As described, the cap 30a may then be advanced into the yoke 18, and the securing handle 160 may rotate slightly as the force between the components increases. At such a time, the drive sleeve 132 may be rotated by the securing handle 160 relative to the body such that the boss 136 is shifted within the notch 84 from the first edge 84a to a second edge 84b. The arc length of the notch 84 and width of the boss 136 may be sized such that the cap 30 is fully secured when the boss 136 contacts the second edge 84b. Alternatively, if it is desirable for the cap 30 to be partially, but not fully, captured or secured, the notch 84 and projection may be sized accordingly to limit the amount of relative rotation between the drive sleeve 132 and the tubular body portion 80 and to provide the tactile indication discussed above.

The sleeve coupling assembly 6 includes a handle sleeve 162, and a knurled sleeve nut 164 (see FIG. 2) in threaded engagement with an inner retainer nut 166. The inner nut 166 further includes internal threads 182 for cooperating with external threads 86 located near the proximal end 80d of the tubular body portion 80. When assembled, the internal threads 182 of the inner nut 166 are secured to the external threads 86 of the tubular body portion 80.

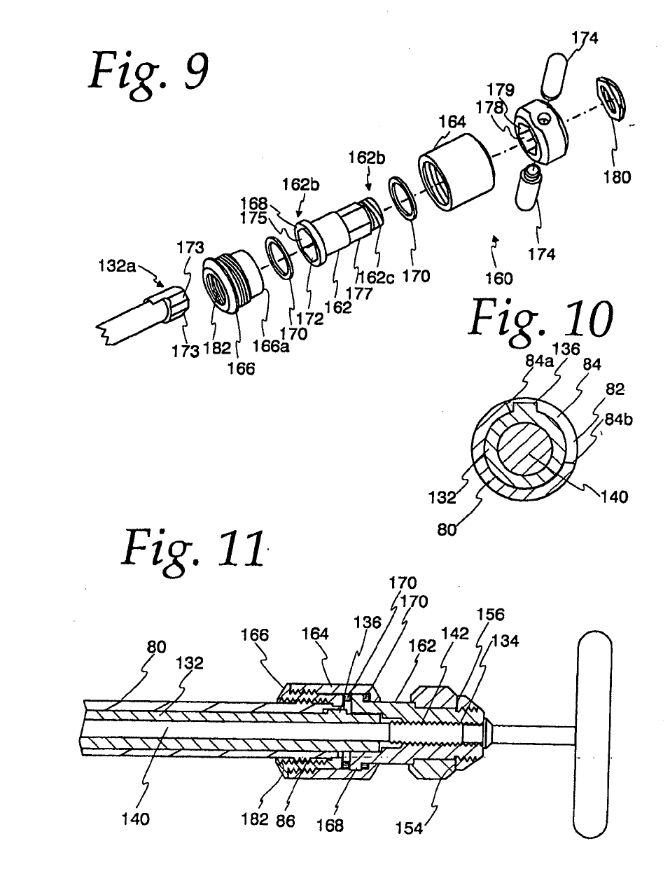

Referring now to FIGS. 9 and 11, a proximal end 162a of the handle sleeve 162 is inserted into the sleeve nut 164, and a shank portion 166a of the inner retainer nut 166 is threaded into the sleeve nut 164. More specifically, the handle sleeve 162 includes an annular flange 168 extending about its distal end 162b, and the sleeve nut 164 and inner nut 166 are threaded together with the flange 168 captured therebetween. Bearing members such as in the form of washers 170 separate the shoulder 168 from each of the sleeve nut 164 and inner nut 166. Preferably, the bearing members are preferably low friction members so that the handle sleeve 162 may rotate freely relative to the sleeve nut and inner nut 166 when they are secured.

The handle sleeve 162 has a central bore 172 through which the drive rod 140 extends. The central bore 172 includes the threaded portion 134 that cooperates with the threaded portion 142 of the drive rod 140 for threadably advancing or retracting the drive rod 140. In addition, the central bore 172 and a proximal end 132a of the drive sleeve 132 include cooperating structure such that the shaft sleeve proximal end 132a may be inserted into the handle sleeve central bore 172 in a predetermined rotational orientation and such that the cooperating structure prevents relative rotation therebetween. As illustrated in FIG. 9, the cooperating structure includes flats 173 and 175 on the drive sleeve proximal end 132a and in the central bore 172, respectively.

As shown in FIG. 9, the handle 160 includes a pair of laterally extending handle grip portions 174. The grip portions 174 extend laterally from opposite sides of a handle cap 176 and may be integral with or otherwise secured to the cap 176. The handle cap 176 includes a central bore 178 with a non-circular inner structure, such as an octagon, for receiving the handle sleeve 162 therein. The proximal end 162a of the handle sleeve 162 has an outer surface for cooperating with the central bore 178 such that the handle sleeve 162 and handle cap 176 are prevented from relative rotation. For instance, the cooperating structures may include flats 177 and 179 on the handle sleeve 162 and in the central bore 178, respectively. A terminal portion 162c of the handle sleeve 162 projects through the handle cap 176 and is secured by a cap nut 180.

In addition, the length of the retainer nut shank 166a extending into the bore of the sleeve nut is sized to enable the tool shaft 120 to be advanced towards yoke 18 the proper distance so that the cap holding flanges 40 are aligned with the recesses 41 in the yoke 18. For relatively minor calibrations, the size or number of the washer bearings 170 may be varied. Specifically, the tool shaft 120 advances to force the cap 30 and spinal rod 12 into the yoke 18, eventually forcing the spinal rod 12 into a seated engagement within the yoke 18. At such a point, the securing handle 160 is rotated so that the drive sleeve 132 and the tool shaft 120 therein are rotated, thus securing the cap 30 by rotating the holding flanges 40 thereon into the recesses 41 of the yoke 18. As described, over-rotation of the drive rod 140 relative to the drive sleeve 132 may cause the threadably engaged drive rod 140 and drive sleeve 132 to bind with each other. On the other hand, under rotation of the drive rod 140 will result in under-advancement of the cap 30 and spinal rod 12 so that the cap 30 cannot at least partially be captured within the yoke 18.

To enable a user to advance the tool shaft 120 without over-rotation of the drive rod 140 or under-advancement of the tool shaft 120, the drive rod shoulder 154 is provided to engage with the shoulder 156 when the tool shaft 120 has been advanced to the proper position. The extent to which drive rod 140 is to be advanced relative to the securing handle 160 is dependent on the position of the shoulder 156 formed on the proximal end of the securing handle 160. However, the position of the shoulder 156 on the securing handle 160 relative to the yoke 18 secured in the jaws 60, 62 is dependent on the amount the securing handle 160 is threaded onto the tubular body portion 80. Due to the threading cooperation of the various components of tool 10, this amount is difficult to predict with the degree of certainty desired, the calibration washers 170 are provided to properly locate the shoulder 156 relative to the clamped yoke 18.

As mentioned above, the bearing washers 170 are located in the securing handle 160. The bearing washers 170 also provide a calibration. For instance, any number or thickness of calibration washer 170 may be included, as desired. In the preferred embodiment, the thickness of each washer 170 is 0.010 inches. Washers 170 placed on a distal side of the shoulder 168 locate the shaft sleeve 162 in a more distal position, thereby locating in a more distal position the sleeve nut 164, handle cap 176, and cap nut 180 on which the shoulder 156 is formed. Similarly, washers 170 placed on a proximal side of the shoulder 168 located the sleeve nut 164 in a more distal position, thereby again locating in a more distal position the handle cap 176, and cap nut 180 on which the shoulder 156 is formed.

It should be noted that, alternative to or in conjunction with using the washers 170 to calibrate the tool 100, the position of the inner nut 166 may be adjusted on the tubular body portion 80. In any event, the inner nut 166 may receive epoxy on its internal threads 182 that mate with the body threads 86 to generally fix the inner nut 166 relative to the tubular body portion 80.

Once the drive rod 140 is advanced to a position where the shoulders 154, 156 engage, the securing handle 160 may then be rotated to rotate the drive sleeve 132 and, accordingly, the tool shaft 120. More specifically, the handle cap 176 and grip portions 174 therefrom may be rotated, thus rotating the handle sleeve 162 located therein. The shoulder 168 located between the calibration washers 170 is free to rotate relative to the inner nut 166, and thus relative to the tubular body portion 80 and jaws 60,62 secured thereto. As the handle sleeve 162 is rotated, the drive sleeve 132 is rotated so that the tool shaft 120 and cap 30 are rotated, thus at least partially capturing the cap 30 within the yoke 18.

Referring now to FIGS. 3a-6b, the operation of the tool 10 to seat the cap 30 and spinal rod 12 within the yoke 18 is sequentially illustrated. In FIGS. 3a, 3b, the tooth 38 of the stationary jaw 60 is inserted into a complementary recess 36 in a wall 22 of the yoke 18. In this manner, the complementary yoke and jaw surfaces 24,70 are mated, and the shoulder 72 of the stationary jaw 60 is against the yoke top surface 26. The lever 92 is in the open, unsecured position such that the movable jaw 62 is also open such that the cap 30 and spinal rod 12 may be received between the jaws 60,62. As illustrated, the spinal rod 12 is positioned against a cap bottom surface 30b, and both are positioned between the jaws 60,62.

The movable jaw 62 is then moved to the closed position, as is shown in FIGS. 4a and 4b. More specifically, the lever 92 is moved towards the tubular body portion 80 and into the closed position. Consequently, the movable jaw 62 moves from the open position to the closed position so that the tooth 38 of the movable jaw 62 is received within a recess 36 in a second wall 22a of the yoke 18. Again, complementary yoke and jaw surfaces 24,70 of the yoke 18 and movable jaw 62 are mated, and the shoulder 72 of the jaw 62 is against the yoke top surface 26. In this position, the tool 10 and the yoke 18 are in a generally fixed orientation aligned along the axes Y and R of the yoke 18 and tool 10, respectively. At this point, the tool 10 is in a position and configuration for advancing the cap 30 and spinal rod 12 into the yoke 18.

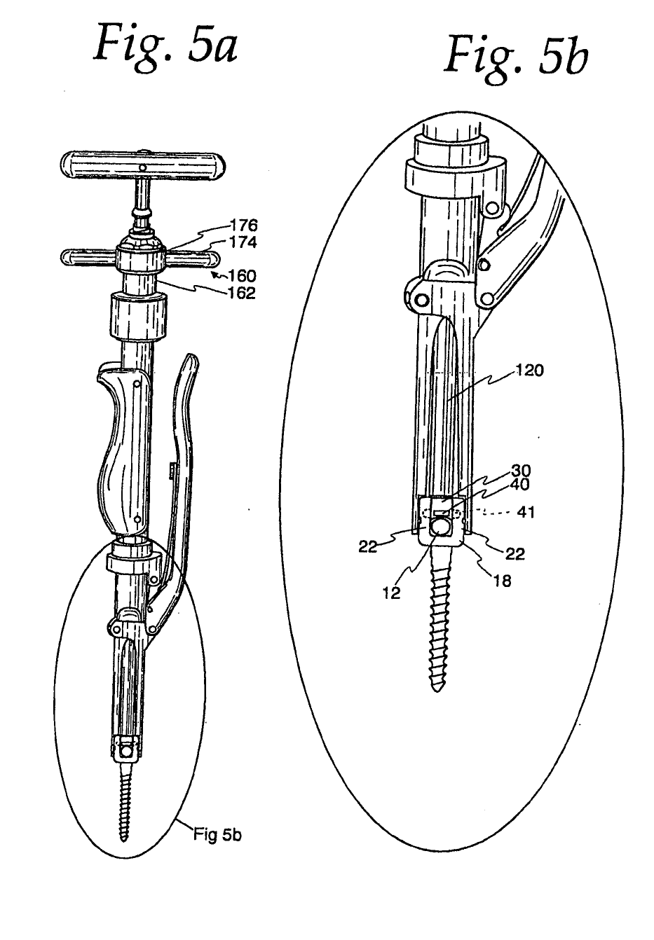

As can be seen in FIGS. 5a and 5b, the tool shaft 120 is advanced linearly along the axis Y of the yoke 18 in order to advance the cap 30 and the spinal rod 12 to a position between the walls 22 of the yoke 18. When fully advanced, the holding flanges 40 of the cap 30 are positioned within the yoke 18 so as to align with the recesses 41 of the yoke walls 22. To at least partially secure the cap 30 within the yoke 18, the cap 30 is then rotated, as is depicted in FIGS. 6a and 6b. To achieve this, the tool shaft 120 is rotated, as described herein, and the holding flanges 40 of the rotated cap 30 are received in the recesses 41 of the yoke 18. The cap 30 may be rotated to a final position to seat the cap 30 and spinal rod 12 within the yoke 18 such that the holding flanges 40 are fully located within the recesses 41 of the yoke 18, or may be rotated to a position sufficient to partially secure the cap 30 within the yoke 18 so that a surgeon may secure the spinal rod 12 to a plurality of yokes 18, and then secure the caps 30 with a separate instrument as a final locking step. To rotate the cap 30 to secure the cap 30, the prongs 174 attached to the handle cap 176 are rotated, thereby rotating the drive sleeve 132 connected thereto, the tool shaft 120 within the drive sleeve 132, and the cap 30 attached to the drive end of tool shaft 120.

In order to be reusable, the tool 10 may, preferably, be suitably cleaned and sterilized. As described, the tool 10 includes a number of crevices, cooperating components, threads, and cavities into which organic tissue may become lodged. It is known that cleaning and sterilization of surgical instruments benefits from being able to dislodge foreign matter from the instruments and components. Accordingly, the preferred embodiment of the tool 10 is easily assembled and disassembled. For instance, in the present embodiment, the drive rod assembly 3 may be removed from the sleeve coupling assembly 6, which in turn may be removed from the tubular body portion 80.

The disassembly may begin with the sleeve coupling assembly 6. The inner nut 166 and nut sleeve 164 may be threadably disengaged by counter-rotating the nut sleeve 164 relative to the inner nut 166. If the inner nut is not secured, such as by the above-described epoxy, to the tubular body portion 80, the internal threads 182 of the inner nut 166 may be threadably disengaged from the external threads 86 of the tubular body portion 80 by counter-rotating the inner nut 166 relative to the tubular body portion 80. The securing handle 160 may then be removed from the tubular body portion 80, along with the drive rod assembly 3 and its drive rod 140, tool shaft 120, and drive handle 146. The drive sleeve 140 may then be removed from the throughbore 82. The sleeve coupling assembly 6 may be disassembled by removing the cap nut 180.

The drive handle 146 may be counter-rotated such that it is threadably disengaged from the handle sleeve 162 of the sleeve coupling assembly 6. Consequently, the drive rod 140 and tool shaft 120 may be removed from the securing handle 160. The securing handle 160 may also be disassembled, if desired. The grip 11 may be removed from the tubular body portion 80 be removing or loosening the set screws 13. In this manner, the tool 10 may be autoclaved or cleaned and sterilized.

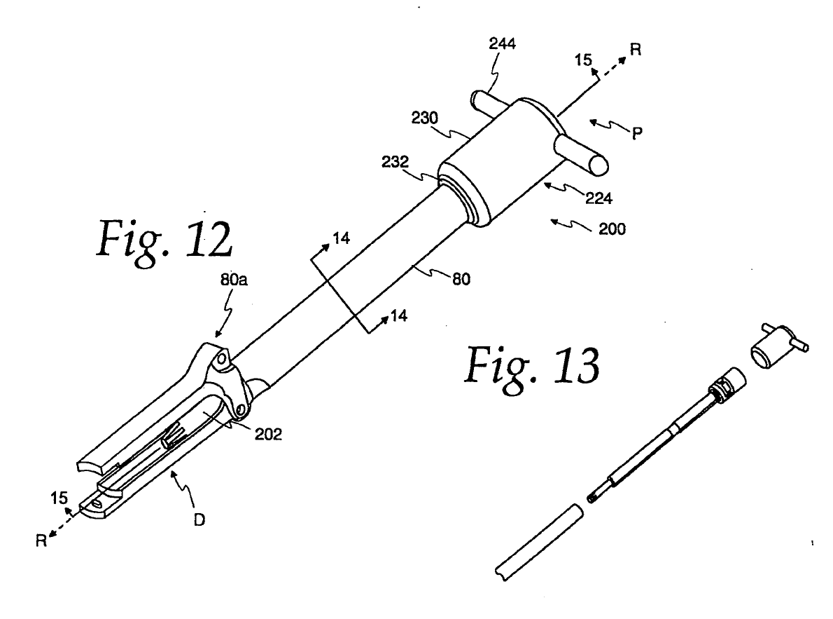

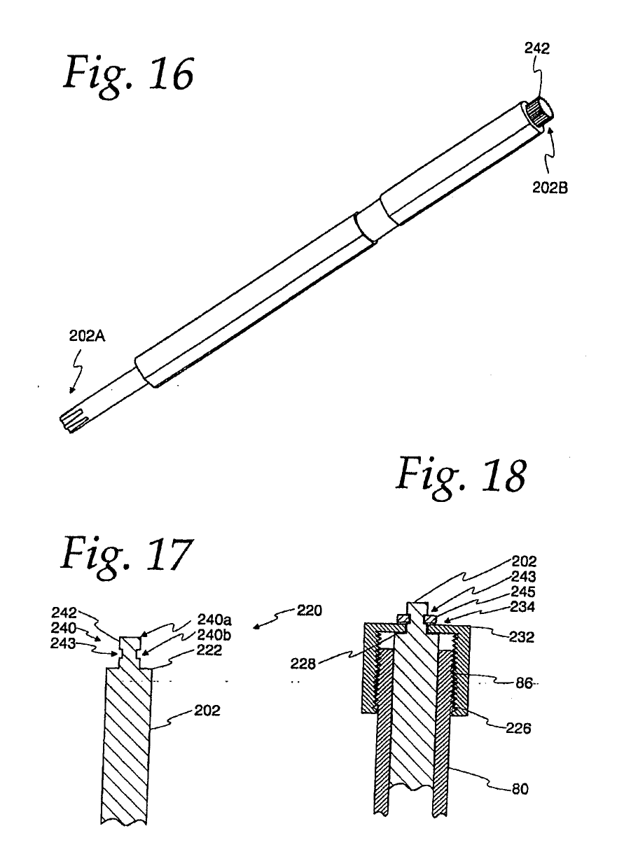

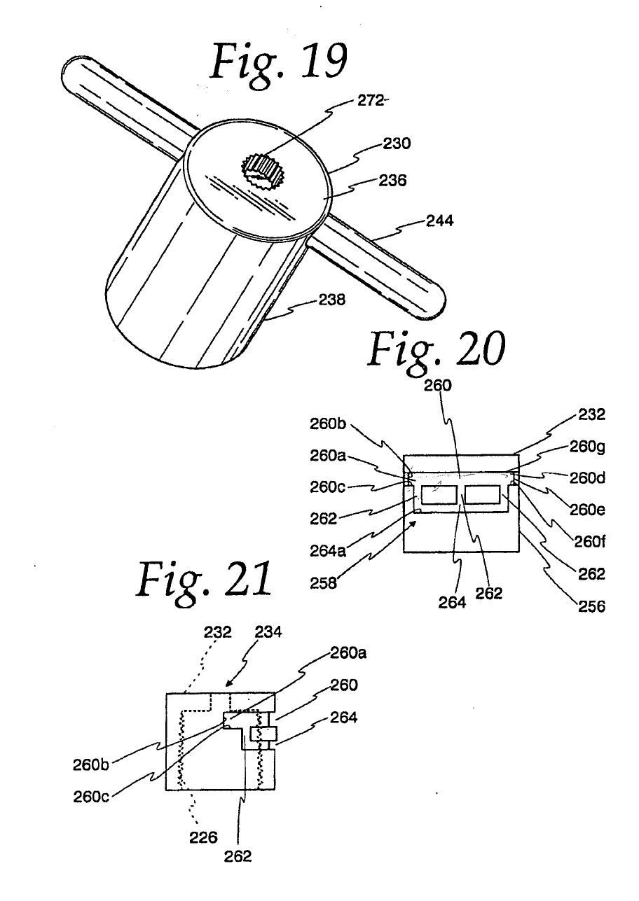

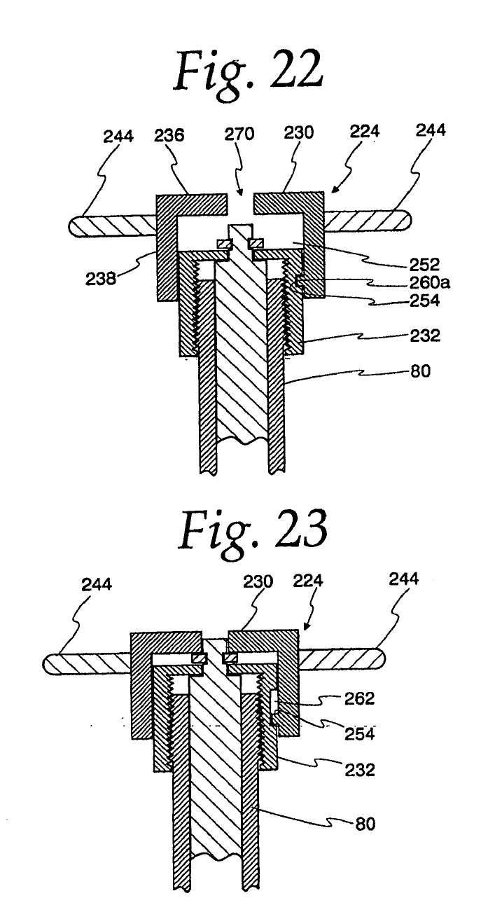

A second form of a persuader tool 200, depicted in FIGS. 12-23, includes a system for advancing a drive rod 202 and cap 30 linearly until the cap 30 is aligned with recesses 41 in the yoke 18, whereupon the cap 30 and drive rod 202 are rotated to capture at least partially the cap 30 within the yoke 18. The tool 200 includes the jaws 60,62, lever 92, and tubular body portion 80, as generally described above.

As discussed, the tubular body portion 80 includes a generally cylindrical throughbore 82, a distal end 80a, a proximal end 80d, and external threads 86 near the proximal end 80d. The drive rod 202 is received within the throughbore 82 such that the drive rod 202 may be advanced or retracted linearly within the throughbore 82 but is prevented from rotating relative to the tubular body portion 80 within the throughbore 80.

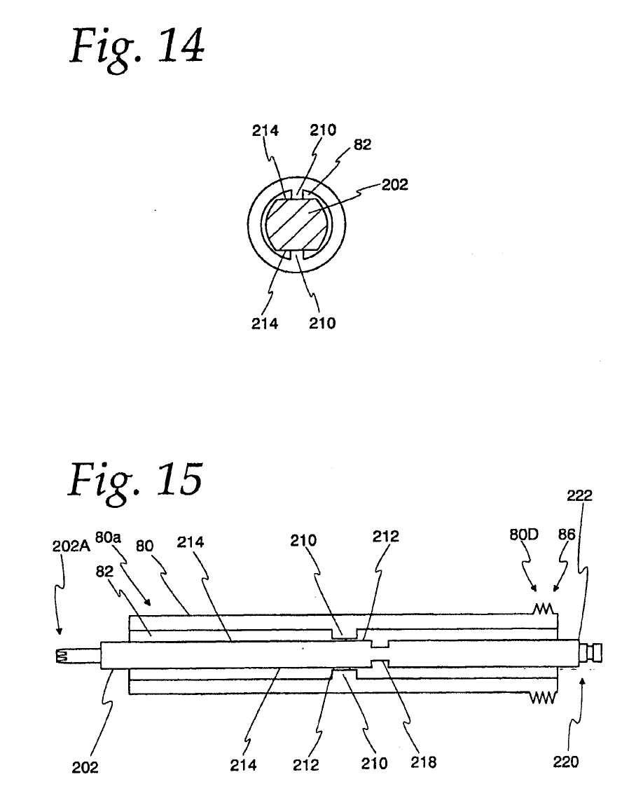

As can best be seen in FIGS. 14 and 15, the drive rod 202 and tubular body portion 80 are provided with cooperating structure to prevent rotational relative movement therebetween as the drive rod 202 is advanced. In the present embodiment, the tubular body portion 80 includes one or more internal protrusions 210 extending into the throughbore 82 and having preferably generally flat surfaces 212. The drive rod 202 is generally cylindrical with truncated sides to form flat surfaces 214 that abut and may slide along the flat surfaces 212 of the protrusions 210. In this manner, the flat surfaces 212,214 cooperate to permit longitudinal translation of the drive rod 202 relative to the tubular body portion 80 while generally preventing rotational relative movement therebetween.

As discussed above, the drive rod 202 is rotated once the cap 30 is generally located within the yoke 18 to at least partial secure the cap 30 therein. In order to provide for this rotation, the drive rod 202 has a reduced portion 218. Once the drive rod 202 has advanced sufficiently such that the cap 30 is located within the yoke 18 such that its holding flanges 40 are aligned with recesses 41 of the yoke 18, the reduced portion 218 of the drive rod 202 is positioned and aligned with the flat surfaces 212 of the tubular body portion 80 such that the flat surfaces 214 of the drive rod 202 are clear of the protrusions 210. Consequently, the protrusions 210 do not hinder the rotation of the drive rod 202 relative to the tubular body portion 80 at this position, and the cap 30 can be at least partially secured by rotation of the drive rod 202.

The drive rod 202 includes a drive end 202a for cooperating with the drive recess 122 of the cap 30. In the present embodiment, the drive rod 202 further includes a proximal end 220 with a shoulder 222 generally directed towards the user. In order to advance the drive rod 202, a sleeve coupling assembly 224 is provided for both drive and securing the cap 30.

Preferably, the sleeve coupling assembly 224 includes a rotatable drive member for drive the drive rod 202, such as in the form of a lock sleeve 230, and a position sleeve 232 located within and cooperating with the lock sleeve 230. The sleeve coupling assembly 224 has internal threads 226 for threadably engaging the external threads 86 of the tubular body portion 80. In this manner, the sleeve coupling assembly 224 may be rotational advanced or retracted relative to the tubular body portion 80. The sleeve coupling assembly 224 further includes an internal shoulder 228 and, as the sleeve coupling assembly 224 advances, the shoulder 228 abuts the shoulder 222 of the drive rod 202 such that the sleeve coupling assembly 224 and drive rod 202 advance together.

The position sleeve 232 includes a central bore 234 around which the internal shoulder 228 is located. Extending from the tool shaft shoulder 222 and through the central bore 234 of the position sleeve 232 is a stepped engagement 240 including a proximal, securing portion 240a and a distal, retention portion 240b, as best can be seen in see FIG. 17. The securing portion 240a includes a portion 242 with gear teeth or splines located radially therearound, and the retention portion 240b is generally cylindrical and includes a recess 243 for receiving a retaining clip 245. The retaining clip permits the drive rod 202 to rotate relative to the position sleeve 232 while generally attaching the drive rod 202 to the position sleeve 323.

In addition, the sleeve coupling assembly 224 may be counter-rotated such that, for instance, the persuader 200 may be released from the yoke 18 after the cap 30 has at least been partially secured. This counter-rotation retracts the position sleeve 232 which, in turn, linearly retracts the drive rod 202 connected thereto by the retaining clip.

The lock sleeve 230 and position sleeve 232, as well as the engagement 240, cooperate in a plurality of positions for advancing, rotating, and retracting the drive rod 202. The lock sleeve 230 includes handle prongs 244, an end wall 236, and a cylindrical wall 238. The end wall 236 and cylindrical wall 238 define a central cavity 252 within which the position sleeve 232 is received. The lock sleeve 230 further includes an internal protrusion 254 extending into the central cavity 252. The position sleeve 232 includes an external surface 256 with structural relief 258 for cooperating with the protrusion 254 of the lock sleeve 230.

The relief 258 includes a recess in the form of a drive groove 260 and a securing groove 264, each generally aligned as a circumferential recess on the surface of the position sleeve 232, and a plurality of channels 262 extending in a direction parallel to the axis R of the persuader 200 from and between the drive groove 260 and securing groove 264. A portion 260a of the drive groove 260 extends beyond the channels 262 in the direction of rotation. During operation, a user may rotate the lock sleeve 230 with the protrusion 254 located within the portion 260a and contacting front edge 260b.

During operation, there is a significant amount of compressive force that builds between the advancing drive rod 202, the cap 30, the spinal rod 12, and the yoke 18. This may require, or merely inspire, a user to apply pressure in order to rotate the sleeve coupling assembly 224. In the event the user applies pressure to the lock sleeve 230 during rotation, the protrusion 254 may be positioned in the drive groove 260 such that it also contacts a distal edge 260c therein.

To counter-rotate the sleeve coupling assembly 224 for drive rod 202 extraction, a second portion 260d is provided with a rear edge 260e against which the protrusion 254 is engaged. Because of the described force build-up within the system, the position sleeve 232 is forced towards the user and against the lock sleeve 230, which can cause pressure and friction on the threads 86,226 of the tubular body portion 80 and lock sleeve 230. For this reason, retraction of the drive rod 202 may also benefit from a user applying force while counter-rotating the sleeve coupling assembly 224. For this reason, a distal edge 260f is provided in contact with the protrusion 254 during counter-rotation. Once the pressure or force is released, the portion 260d has a proximal edge 260g against which the protrusion 254 contacts to withdraw the position sleeve 232 rotationally connected to the drive rod 202 by the retaining clip 245.

Rotation of the lock sleeve 230 causes rotation of the position sleeve 232 when the protrusion is located at the distal edge 260c or rear edge 260e of the position sleeve 232. Movement of the lock sleeve 230 within the drive groove 260 and between the edges 260c, 260e does not cause rotation of the position sleeve 232 or drive rod 202. Therefore, the lock sleeve 230 may be adjustably positioned between the portions 260a and 260d without affecting the position of the drive rod 202 or position sleeve 232. If necessary, the position sleeve 232 may be provided with a surface or structure (not shown) for manually immobilizing the position sleeve 232 during adjustment of the position of the lock sleeve 230 relative thereto.

During advancement of the cap 30, when the drive rod 202 does not rotate, the lock sleeve 230 rotates freely relative to the drive rod 202. Once the cap 30 has been sufficiently advanced within the yoke 18, the drive rod 202 is preferably rotated to at least partially secure the cap 30 therein. To achieve this, the lock sleeve 230 may be locked or unlocked with the drive rod 202.

In the present embodiment, the lock sleeve 230 is generally positioned such that the protrusion 254 is located within the drive groove 260 during advancement of the cap 30 and spinal rod 12 into the yoke 18. In order to lock the lock sleeve 230 with the drive rod 202 so that the drive rod 202 may be rotated to at least partially capture or secure the cap 30, the lock sleeve 230 is positioned such that the protrusions 254 is located in the securing groove 264. More specifically, the protrusion 254 is aligned with one of the channels 262, and the lock sleeve 230 is moved distally relative to the position sleeve 232, with the protrusion 254 following one of the channels 262, until the protrusion 254 contacts a distal edge 264a of the securing groove 264.

The end wall 236 of the lock sleeve 230 includes an opening 270 having internally disposed splines or gears 272. As the lock sleeve 230 is moved towards the distal end D of the persuader 200, the opening 270 of the end wall 236 receives the securing portion 240a of the engagement 240. More specifically, the gears 272 of the opening 270 align and mesh with the gears or splines of the central portion 242 of the engagement 240. Once the gears 272 have engaged with the central portion 242, the lock sleeve 230 may be rotated to rotate the drive rod 202, thus rotating the cap 30 to an at least partially captured or secured position.

The persuader 200 may be disassembled in a manner similar to that described above for the tool 100. In this embodiment, the lock sleeve 230 may be counter-rotated and un-threaded from the threads 86 of the tubular body portion 80 of the persuader 200. The position sleeve 232 may then be removed from the cavity 252 of the lock sleeve 230. Finally, the position sleeve 232 may be separated from the drive rod 202 by removing the retaining clip.

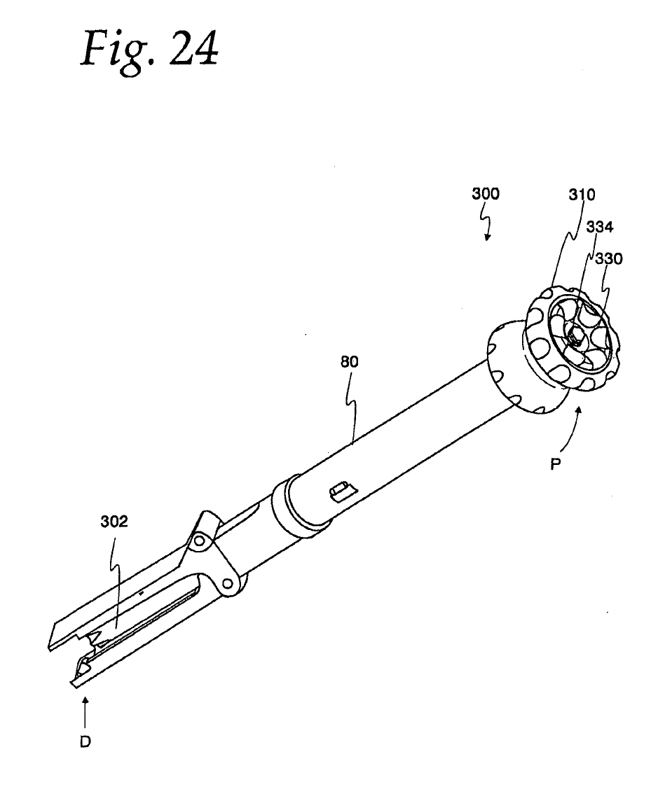

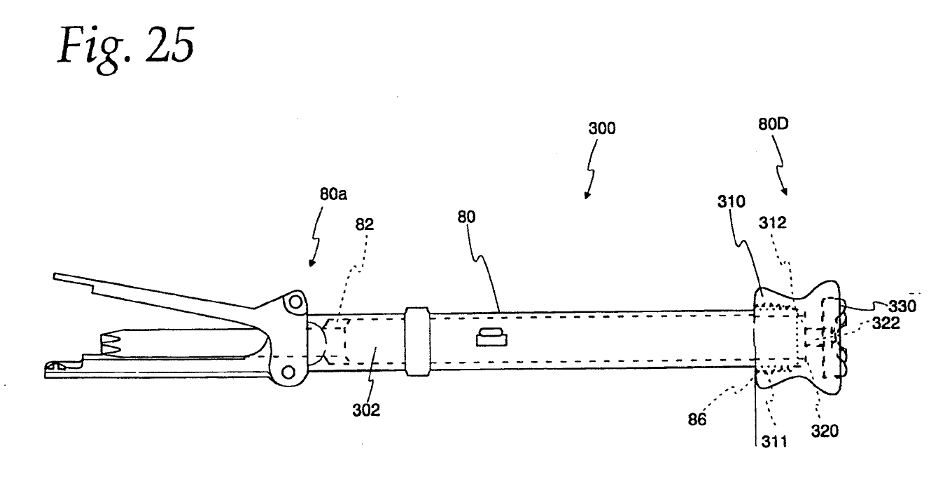

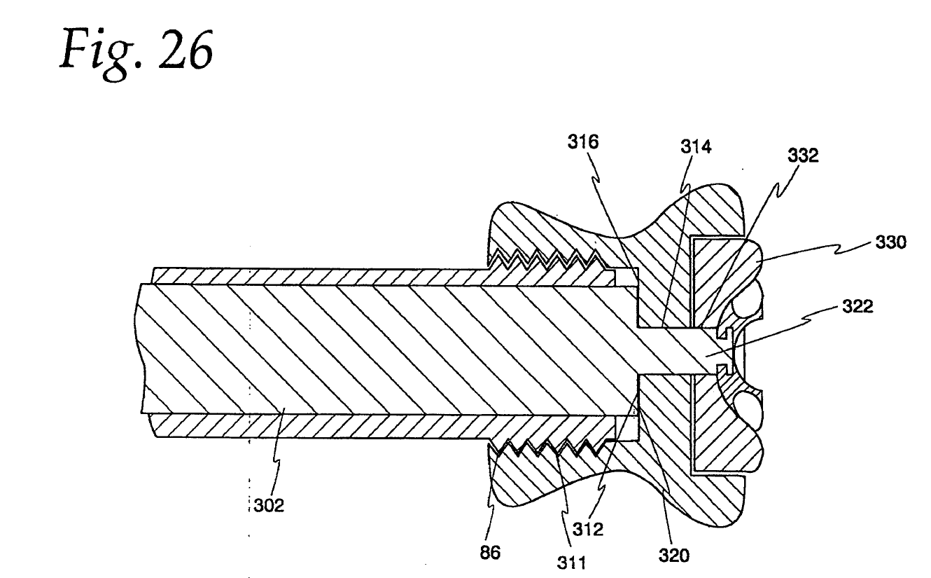

A further form of a persuader 300 including a system for advancing a tool shaft 302 and cap 30 linearly until the cap 30 is aligned with recesses 41 in the yoke 18, whereupon the cap 30 and tool shaft 302 are rotated to capture at least partially the cap 30 within the yoke 18 is depicted in FIGS. 24-26. The persuader 300 includes the jaws 60,62, lever 92, and tubular body portion 80, as generally described above. Again, the tubular body portion 80 includes the generally cylindrical throughbore 82, distal end 80a, proximal end 80d, and external threads 86 near the proximal end 80d. The tool shaft 302 is received within the throughbore 82 such that the tool shaft 302 may be advanced or retracted linearly within the throughbore 82.

The persuader 300 includes a drive grip 310 including internal threads 311 to mate with the external threads 86 of the tubular body portion 80. As the drive grip 310 is advanced on the tubular body portion 80, the drive grip 310 forces the tool shaft 302 to advance towards the yoke 18. In this manner, the cap 30 and spinal rod 12 are driven into a seated arrangement.

The drive grip 310 includes an end wall 316 forming an internal shoulder 312 and having a central port 314 formed therein. The tool shaft 302 includes a shoulder 320 and a securing post 322. As the drive grip 310 is advanced, the grip shoulder 312 contacts the tool shaft shoulder 320 for forcing the tool shaft 302 towards the yoke 18. The securing post 322 of the tool shaft 302 passes through the central port 314 of the drive grip 310, and the drive grip 310 and tool shaft 302 are free to rotate relative to each other.

As stated above, it is preferable to prevent the tool shaft 302 from rotating until the cap 30 has been directed within the yoke 18 and is in a position to be rotated for at least partial securement or capture within the yoke 18. To prevent this rotation during rotation of the drive grip 310 and advancement of the tool shaft 302, a securing grip 330 is provided. The securing grip 330 has an internal bore 332 with structure cooperating with the securing post 322 of the tool shaft 302 such that the securing grip 330 and tool shaft 302 are generally prevented from relative rotation. As the drive grip 310 is rotated, the securing grip 330 is held stationary by manual force. The securing grip 330 is held to the securing post 322 with a retaining clip 334 such that, during counter-rotation of the drive grip 310 such that drive grip 310 is threadably retracted, the tool shaft 302 is linearly retracted, as described above. Rotation of only the securing grip 330 rotates the tool shaft 302 without linear advancement.

Again, disassembly is simple. The drive grip 310 may be un-threaded and removed from the tubular body portion 80, which also withdraws the tool shaft 302 from the body throughbore 82. The retaining clip 334 may be removed from the securing post 322, and the drive grip 310 and securing grip 330 may be removed from the tool shaft 302.

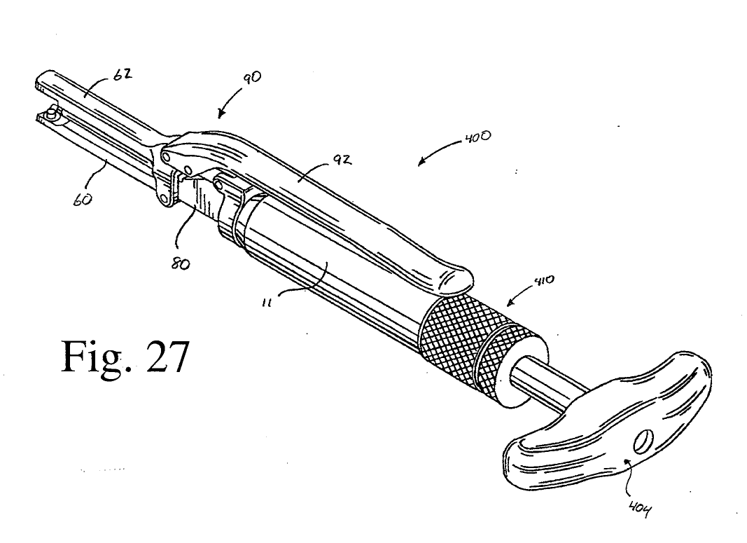

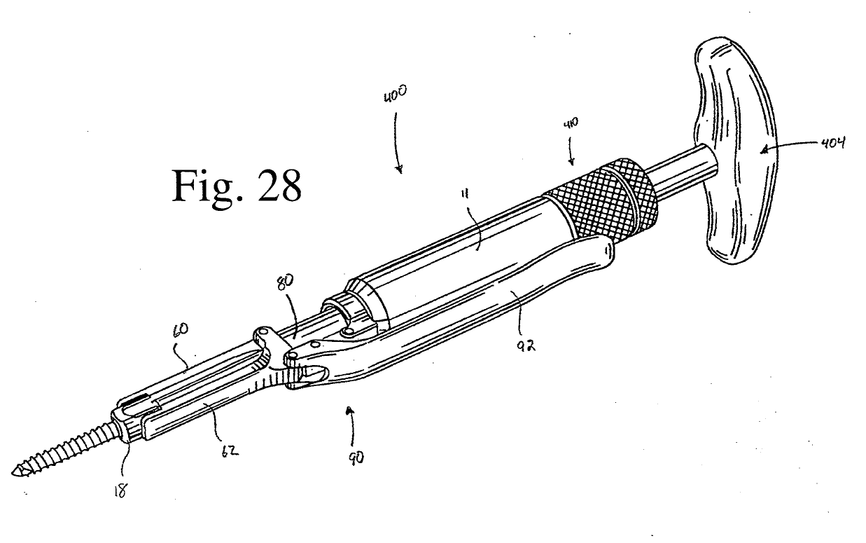

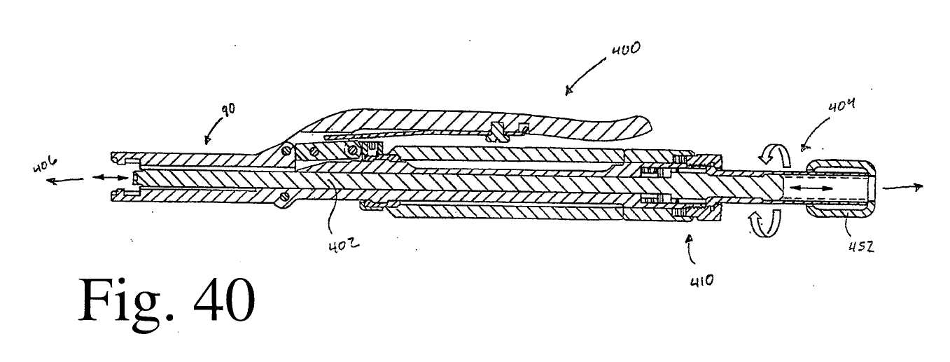

Another form of a persuader tool apparatus 400 is illustrated in FIGS. 27 to 42. The persuader apparatus 400 includes a system to advance a drive rod 402 linearly along an elongate body 80 of the tool apparatus 400 and to rotate the drive rod 402 relative to the body 80 using a single actuator handle 404. As a result, the same rotary motion of the single handle 404 first positions the lock device or cap assembly 30 and spinal rod 12 in the yoke coupling device 18 and also rotates at least a portion of the cap 30 to capture the cap 30 within the yoke 18. Similar to the other embodiments, the tool apparatus 400 also preferably includes a clamping head or the clamping subassembly 90 that includes the jaws 60 and 62, the clamp actuator or lever 92, and the tubular body portion 80 as generally described above. Accordingly, only the differences from the previous embodiments will be described in detail for the persuader tool 400.

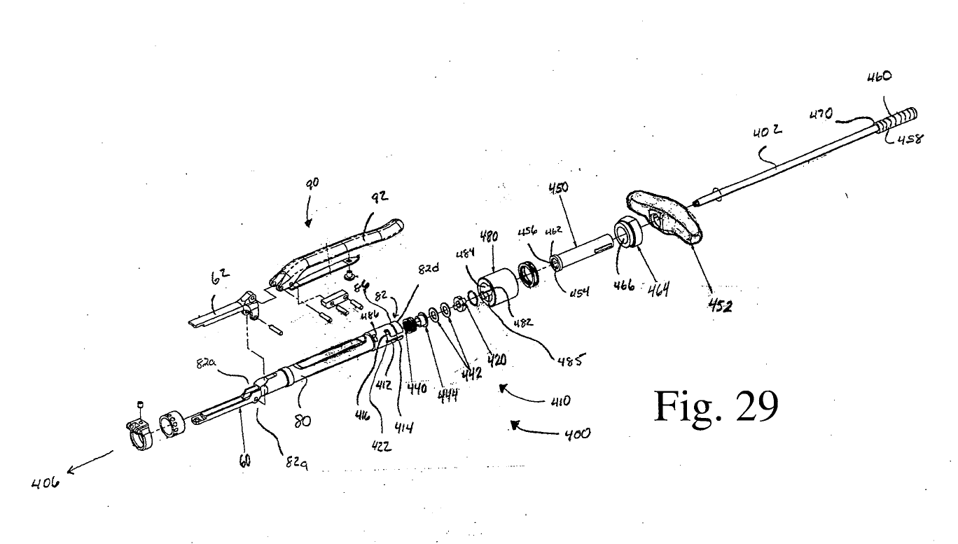

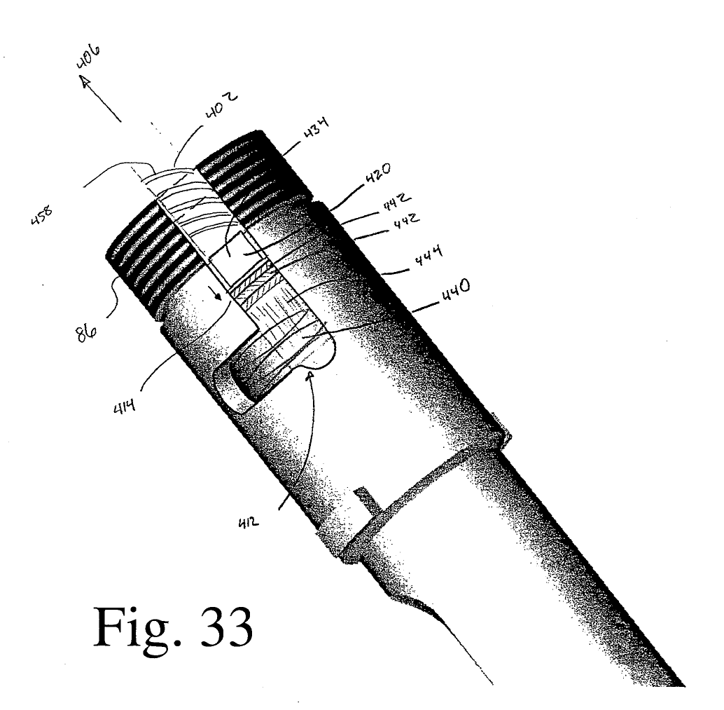

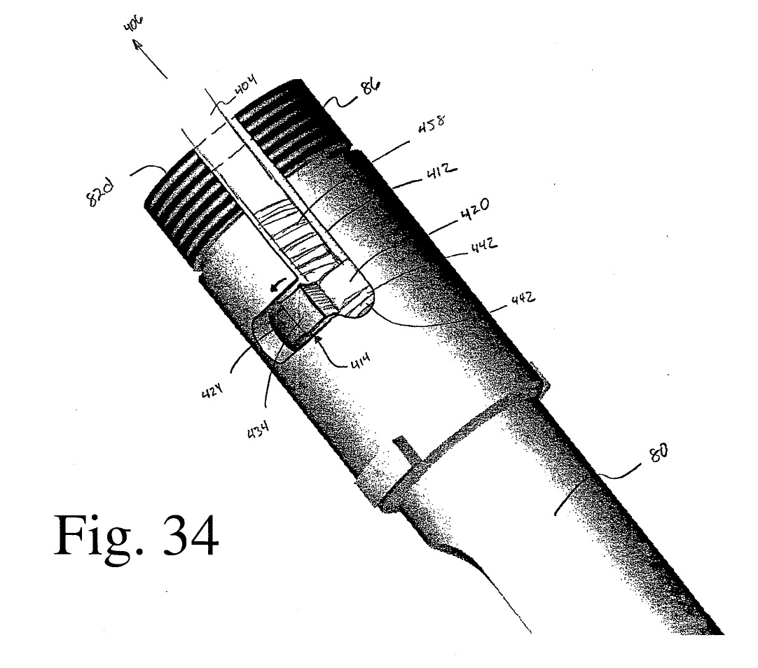

More specifically, the tool apparatus 400 includes the single actuator handle 404 that is operable to both advance the drive rod 402 linearly along a longitudinal axis 406 of the tool apparatus 400 to drive the cap assembly 30 and spinal rod 12 into the yoke 18 and also to rotate the drive rod 402 so that at least a portion of the cap assembly 30 can be locked in the yoke coupling device 18. To this end, the tool apparatus 400 includes a coupling device or switching device 410, which is best illustrated in FIG. 29, operable to automatically shift from an advancing configuration to a rotary configuration. In the advancing configuration of the coupling device 410 (FIG. 33), turning of the single actuator handle 404 linearly advances the drive rod 402, without substantial rotation thereof, along the longitudinal axis 406 in order to advance the cap assembly 30 and spinal rod 12 into the coupling member 18. In the rotary configuration of the switching device 410 (FIG. 34), turning of the same, single actuator handle 404 rotates the drive rod 402, without substantial linear advancement thereof, to rotate at least a portion of the cap assembly 30 in order to lock the cap assembly 30 relative to the yoke member 18. The single handle 404 is advantageous over prior persuader tools because the surgeon does not need to manipulate two handles to both linearly advance and rotate the drive rod 402. The single handle 404 accomplishes both motions through the shifting of the switching device 410.

Turning to more of the details, the tubular body portion 80 includes the generally cylindrical throughbore 82, the distal end 82a, the proximal end 80d, and external threads 86 near the proximal end 82d similar to the other embodiments. The drive rod 402 is received in the throughbore 82 such that the drive rod 402 may be advanced or retracted linearly and also rotated depending on the position of the coupling device 410. To effect the shifting, the switching device 410 is arranged and configured to couple the proximal end 82d of the tubular body 80 to the handle 404.

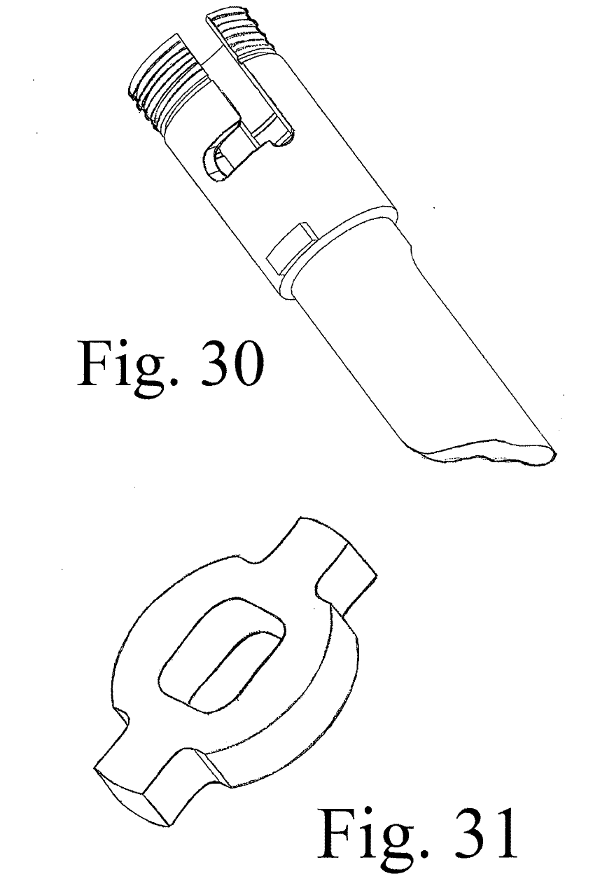

Referring to FIG. 30, a portion of the switching device 410 includes at least one, and preferably two, guide slots 412 extending through the proximal end 82d of the tubular body 80. In the illustrated embodiment, each guide slot 412 is generally L-shaped so that it includes both a longer axial portion 414 extending along the longitudinal axis 406 of the tubular body 80 and a shorter transverse or circumferential portion 416 that extends generally orthogonal to the longitudinal axis 406 of the tubular body 80 similar to a bayonet connection. The two guide slots 412 are on opposite sides of the tubular body 80 with each transverse portion 416 extending in the same circumferential direction about the tubular body 80 so that the transverse portions 416 are not extending toward each other.

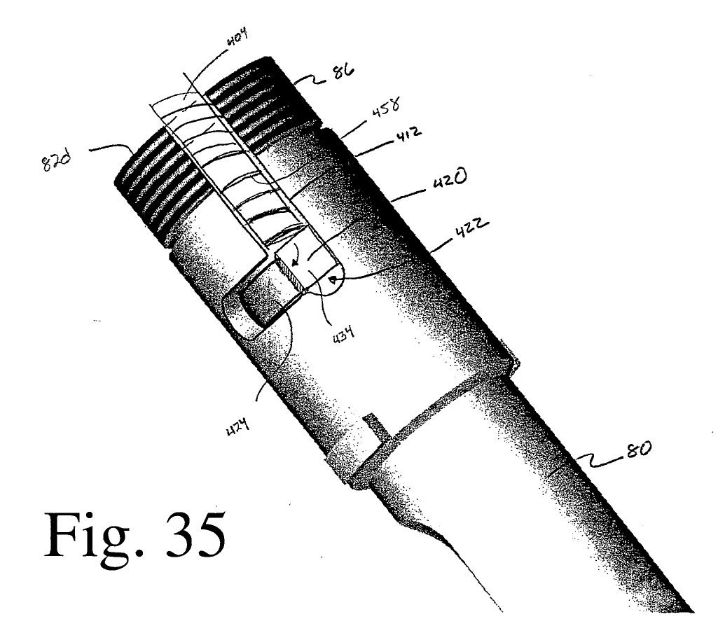

To shift between the two configurations, the switching device 410 includes a switching insert, lock bar or follower member 420. A portion of the follower member 420 is received within the guide slot 412 and is operable, depending on the axial position thereof, to slide in either the axial slot portion 414 or the transverse slot portion 416 upon rotation of the handle 404. That is, depending on the position of the follower member 420, the turning of the handle 404 will either linearly advance the drive rod 402 or rotate the drive rod 402 as explained more fully below. For example, when the follower member 420 is in the axial slot portion 414 (FIG. 33), the coupling device 410 is in the advancing configuration where the follower member 420 translates the turning motion of the handle 404 to linearly advance the drive member 404 along the axis 406 of the tubular body 80 without substantial rotation thereof. On the other hand, when the follower member 420 is in the transverse slot portion 416 (FIG. 34), the switching device 410 is in the rotary configuration where the follower member 420 translates the continued turning motion of the same handle 404 to rotate the drive rod 402 without substantial linear motion thereof. To switch between the two configurations, the slot 412 includes a transition portion 422 where the rotary motion of the handle 404 moves the follower member 420 from the axial slot portion 414 to the slot transverse portion 416 (see, e.g., FIG. 35).

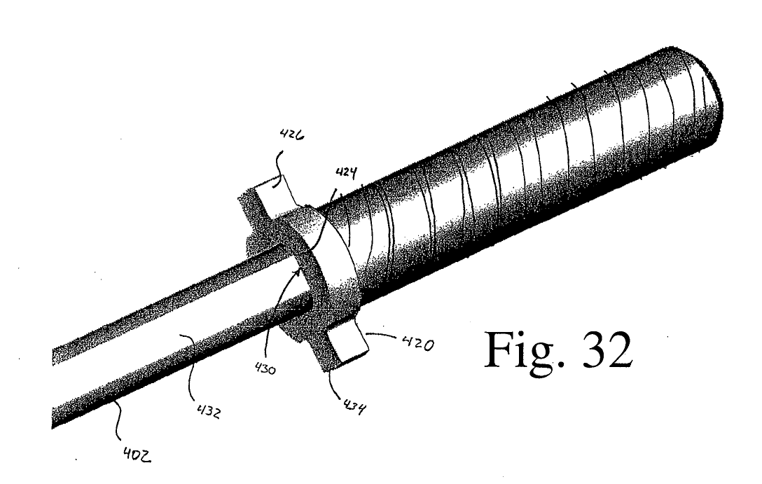

Turning to FIGS. 31 and 32, to permit the turning motion of the handle 404 to either linearly advance or rotate the drive rod 402, the follower member 420 includes a generally annular, main body 424 that is slideably received within the tubular body throughbore 82 and has extensions or radial tabs 426 that protrude outwardly from the main body 242 into the guide slots 412. The drive rod 402 is inserted through a central throughbore 428 of the follower member main body 424 and is prevented from rotating relative to the follower member 420 via cooperating flats 430 of the follower member throughbore 428 and drive rod 402. That is, facing flat portions 431 of the follower member throughbore 428 cooperate with opposing flat portions 432 on the drive rod 402 to prevent relative rotation therebetween.

In one form, the radial tabs 426 are a pair of wings 434 that extend outwardly from opposing sides of the insert main body 424. As best shown in FIGS. 33 to 35, the wings 434 are sized and shaped to be received in and slide through the guide slot 412 in response to a turning motion of the handle 404. When the wings 432 are in the axial portion 414 of the slot 412, the coupling device is in the advancing configuration, and when the wings 432 are in the transverse portion 416 of the slot 412, the switching device 410 is in the rotary configuration.

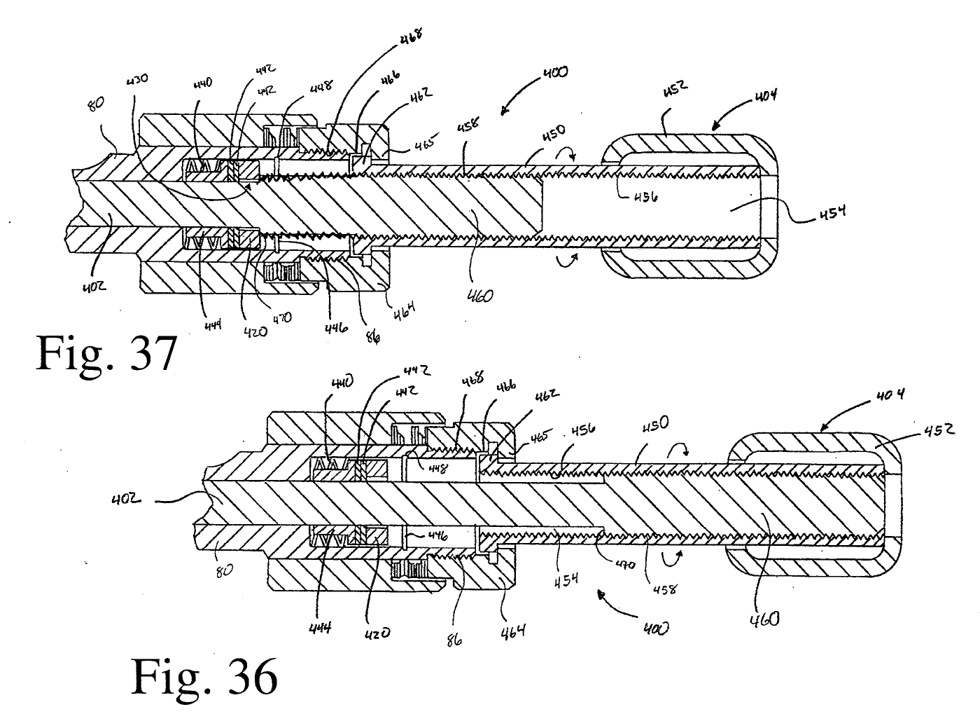

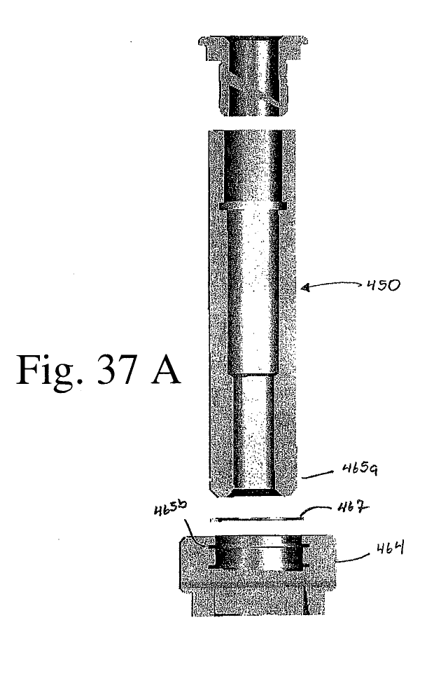

Turning back to FIG. 29, additional components of the switching device 410 are illustrated. To provide some resistance to movement of the follower member 420, the switching device 410 further includes a bias member 440 in the form of a coil spring that helps provide a controlled movement of the follower member 420 through the slot portions 414 and 416. In between the bias member 440 and the follower member 420, there is preferably included a pair of washers 442 to provide a stable engagement surface for the bias member 440 against the follower member 420. Additionally, a spacer 444 may also be included to help centrally position the bias member 440 along the drive rod 402 and insert 440 so as to provide a consistent resistance force circumferentially around the drive rod 402. To hold the follower member 420 on the drive rod 402, a retainer clip 446 may also be included that is received in a circumferential slot 448 formed in the throughbore 82 (FIGS. 36 and 37).

In use, turning of the handle 404 causes the drive rod 402 to either advance linearly along the longitudinal axis 406 of the tubular body 80 or rotate about the axis 406. To this end, as best shown in FIGS. 29 and 36-37, the handle 404 includes a handle housing 450 for rotatably coupling to the drive rod 402 and a T-grip 452 fixed to the housing 450 for the surgeon to grasp and turn the drive rod 402. The handle housing 450 includes a bore 454 having an internal thread 456 for threadably mating with an external thread 458 located on a proximate end 460 of the drive rod 402. Opposite the T-grip 452, the housing 450 includes an annular, abutment flange 462 that is arranged and configured to contact the distal end 82d of the tubular body 80 when the handle 404 is assembled to the tubular body 80 through a locking bearing cap 464. The locking bearing cap 464 secures the handle 404 to the tubular body but permits the handle 404 to turn relative to the locking bearing cap 464 and body 80. Alternatively, the handle housing 450 can be rotatively secured to the locking bearing cap 464 via two mating grooves 465a and 46513 and a snap ring 467 as shown in FIG. 37A. Groove 465a is formed on the outer surface of the handle housing 450 and groove 46513 is formed in an inner bore of the locking bearing cap 464. When assembled, the snap ring 467 is received in both grooves 465a and 46513.

The locking bearing cap 464 includes a central bore 466 having an internal thread 468 that is configured to threadably mate with the external thread 86 on the distal end 82d of the tubular body 80 as best shown in FIGS. 36 and 37. The locking bearing cap 464 secures the flange 462 against the distal end 82d of the tubular body 80 by securing the abutment flange 462 between a radially inwardly projecting annular lip 465 of the locking bearing cap 464 and the distal end 82d of the tubular body 80. In addition, the central bore 466 has a diameter sufficient to permit the handle housing 450 received in the central bore 466 to rotate relative to the locking bearing cap 464. The locking bearing cap 464 also provides additional security in addition to the retainer 446 to hold the follower member 420 within the slot 412 and the throughbore 82.

With the handle 404 rotatably secured to the tubular body 80 via the locking bearing cap 464 as described above, the turning motion of the T-grip 452 causes the drive rod 402 to initially linearly advance along the longitudinal axis 406 due to the internal threads 456 of the handle housing 450 mating with the external threads 458 of the drive rod 402. That is, with the locking bearing cap 464 secured to the end of the tubular body 80, turning of the handle 404 translates to linear movement of the drive rod 402 through the mating threads 456 and 458. With the coupling device 410 initially in the advancing position (i.e., FIG. 33), the drive rod 402 is generally unable to rotate or turn due to the engaging flats 430 of the follower member 420 and drive rod 402 hindering relative rotation therebetween and the wings 432 of the follower member 420 in the axial slot portion 414 hindering rotation of the follower member 420 relative to the tubular body 80. Therefore, turning of the handle 404 causes the drive rod 402 to slide or linearly advance along the axis 406 of the tubular body 80.

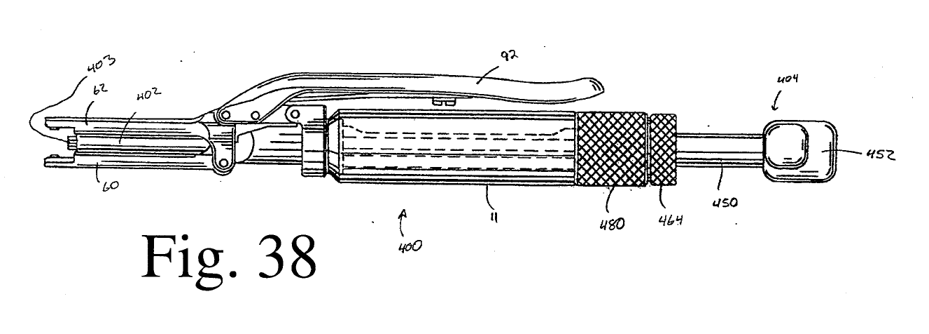

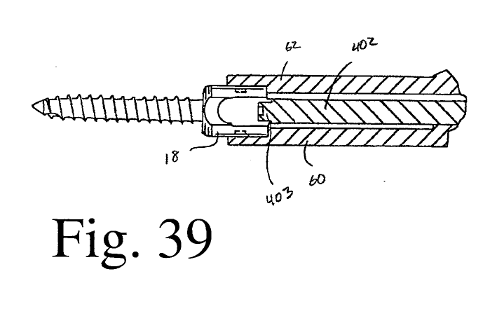

Further turning of the handle 404 causes the drive rod 402 to linearly advance a sufficient amount into the tubular body 80 so that a radially projecting interference 470 (FIGS. 36 and 37), which in this embodiment is a distal end of the external threading 458, engages the upper surface of the follower member 420 and linearly advances the follower member 420 axially in the axial portion 414 of the slot 412. Continued turning of the handle 404 causes the drive rod 402 to advance a predetermined amount, generally without rotation thereof, along the tubular body 80 so that the cap assembly 30 is positioned in at least a first locking position in the yoke coupling member 18 as illustrated in FIGS. 38 and 39. At this same point, the interference 470 has also advanced the follower member 420 in the axial slot portion 414 downwardly to the transition slot portion 422 (FIG. 35) where continued turning of the handle 404 causes the follower member 420 to rotate to the transverse or circumferential portion 416 of the slot (i.e., the switching device 410 shifting to the rotary configuration). Further turning of the handle 404 causes the follower member 420 and drive rod 402 coupled thereto via the flats 430 to rotate a predetermined amount in the transverse slot portion 416, generally without linear advancement thereof, until the insert wings 432 engage a rotary stop 472 (FIG. 30) at the end of the transverse slot portion 416. The rotary stop 472 generally stops further rotation of the drive rod 402, and in one embodiment, limits rotation of the drive rod 402 to less than about 90 degrees. The insert wings 432 also engage an axial stop 474 on the bottom side of the transverse slot portion 416 that also generally stops axial motion of the drive rod 402 in order to position the cap 30 in the desired locking position of the yoke member 18 as shown in FIG. 39.

At this point, to limit the drive rod 402 from rotating in a reverse direction, a lock sleeve 480 may lock the tool apparatus 400 in this axially advanced and rotary advanced position by engaging key slots 482 defined on an inner bore 484 of the lock sleeve 480 into protrusions 486 located on the outer surface of the tubular body 80 as best shown in FIG. 29. In addition, the lock sleeve 480 may also include facing internal slots 485 defined in the inner bore 484 that are positioned to receive the end of the insert wings 432. In this manner, the lock sleeve 480 will also preferably rotate along with the follower member 420 when it shifts from the axial slot portion 414 to the transverse slot portion 416 to provide a visual, audible, and/or tactile indication that the coupling device 410 has shifted from the advancing configuration to the rotary configuration. In addition, the switching device 410 may provide an audible and/or tactile indication that the turning of the handle 404 is complete, such as when the follower member tabs 434 engage the stops 472. Preferably, once the coupling device 410 has shifted to its rotary position, the handle 404 is permitted to turn about another 45 degrees, where the audible and/or tactile indication is provided to notify the surgeon that the cap assembly is locked in the yoke member.

As with the previous embodiments, disassembly of the tool apparatus 400 is simple and quick. For example, in one form, the locking bearing cap 464 may be un-threaded and removed from the tubular body 80, which also permits the withdrawal of the drive rod 402 from the body throughbore 82. In other forms, the drive rod may be unthreaded from the body 80 and handle 404. The components of the switching device 410 may then be removed from the tubular body 80 by sliding them out of the throughbore 82 after the retaining ring 446 has been removed from the throughbore 82. The drive rod 402 may also be unthreaded from the handle housing 450 to further disassemble the tool 400.

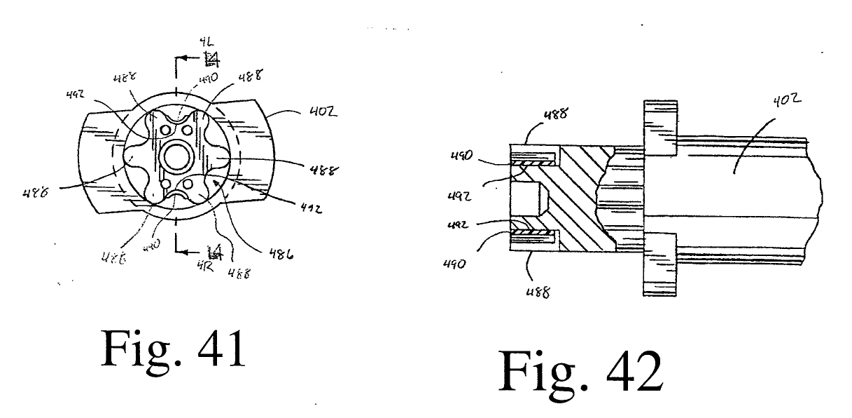

Turning to a distal or drive end 403 of the drive rod 402 as best shown in FIGS. 41 and 42, the drive rod 402 includes a profiled end cap 486 that is arranged and configured to mate with a similarly profiled surface on the upper end of the cap assembly 30 (not shown) via a friction-type fit. In one form, the profiled surface 486 includes a plurality of circumferentially spaced or radially extending lobes 488 formed about the drive end 403 of the drive rod 402. The lobes 488 are configured to be received in a corresponding recess (not shown) formed in the cap assembly 30. Manifestly, the lobes 488 could be on the cap assembly 30 and the recess could be formed on the drive rod 402.

To provide a tight, friction fit with the cap assembly 30, the drive rod 402 may also includes at least one, and preferably two, friction inserts 490 between adjacent lobe portions 488 on opposite sides of the drive rod 402. The friction inserts 490 permit the profiled end 486 of the drive rod 402 to securely hold the cap assembly 30 in a friction-tight fit and generally provide an improved ability to secure the cap 30 to the drive rod 402 over a more traditional, tapered press-fit design of the drive end. In one form, the friction inserts 490 are thermoplastic resin and have a shape to conform to the inner curved transition region 492 between two adjacent lobes 488. Suitable inserts may be provided by Nemcomed, Ltd. (Hicksville, Ohio); however, other inserts and/or materials may also be used with the drive rod 402. It will be appreciated that only two friction inserts 490 are shown in FIGS. 41 and 42, but any number and shape of inserts may be provided to correspond to the profile on the end of the drive rod 402.

The tool apparatus 400, therefore, provides a single actuator handle 404 that advances the drive rod 402 both linearly along the tools longitudinal axis 406 (generally without rotation thereof) and also rotates the drive rod 402 about the tools longitudinal axis 406 (generally without linear advancement thereof) using the same turning motion of the handle 404. Both motions are accomplished via the positioning of the coupling device 410. The tool 400, therefore, provides advantages over prior persuader tools because only one handle is needed to both linearly advance and rotate a cap assembly where prior persuader tools required two handles—one to linearly advance the drive member and one to rotate the drive member.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

1. A surgical tool apparatus for securing a rod and cap assembly in a coupling device, the surgical tool apparatus comprising:
an elongate body;
a clamping head at one end of the body for clamping the coupling device;
a drive member for being advanced along the elongate body to linearly advance the cap assembly in the coupling device and to rotate at least a portion of the cap assembly in the coupling device; and
a single actuator handle operable to advance the drive member for linearly advancing the cap assembly in the coupling device and to rotate the drive member for rotating the cap assembly portion in the coupling device.

2. The surgical tool apparatus of claim 1, further including a switching device operable to shift from an advancing configuration where the single actuator handle linearly advances the drive member generally without rotation to a rotary configuration wherein the single actuator handle rotates the drive member generally without linear advancement.

3. The surgical tool apparatus of claim 2, wherein the switching device further comprises:
a generally L-shaped slot defined in a proximate end of the body having an axial portion extending along a longitudinal axis of the elongate body and a circumferential portion extending about the longitudinal axis; and
a lock bar arranged and configured to slide within the axial portion of the slot with the switching device in the advancing configuration and within the circumferential portion of the slot with the switching device in the rotary configuration.

4. The surgical tool apparatus of claim 3, wherein the circumferential portion of the slot includes an axial stop to limit movement of the drive member along the longitudinal axis when a portion of the lock bar engages the axial stop.

5. The surgical tool apparatus of claim 4, wherein the circumferential portion of the slot includes a rotary stop to limit rotational movement of the drive member when another portion of the lock bar engages the rotary stop.

6. The surgical tool apparatus of claim 5, wherein the switching device includes a bias member positioned to provide resistance to axial movement of the lock bar.

7. The surgical tool apparatus of claim 6, wherein the switching device further includes a sleeve arranged and configured to move with the lock bar so that the sleeve provides a visual indication of the switching device in the rotary configuration.

8. The surgical tool apparatus of claim 7, wherein the drive member includes a radially projecting interference that engages the lock bar to move the lock bar along the longitudinal axis when the switching device is in the advancing configuration.

9. The surgical tool apparatus of claim 8, wherein the radial projecting interference includes a portion of external threading on an outer surface of the drive member.

10. The surgical tool apparatus of claim 9, wherein the generally L-shaped slot includes a transition region where rotation of the single actuator handle causes the switching device to shift from the advancing configuration to the rotary configuration.

11. The surgical tool apparatus of claim 2, wherein the switching device further includes a lock sleeve at the proximate end of the elongate body, the lock sleeve arranged and configured to rotate relative to the drive member with the switching device in the advancing configuration and to rotate the drive rod with the coupling assembly in the rotary configuration.

12. The surgical tool apparatus of claim 11, wherein the switching device further includes a position sleeve concentrically received in the lock sleeve and having a structural relief cooperating with the lock sleeve to rotate the position sleeve and the drive member and for shifting the lock sleeve from the advancing configuration to the rotary configuration.

13. The surgical tool apparatus of claim 12, wherein the structural relief includes a drive recess defined in an outer surface of the position sleeve, a securing recess defined in the outer surface of the position sleeve and spaced axially from drive recess, and at least one channel recess extending therebetween.

14. The surgical tool apparatus of claim 13, wherein the lock sleeve includes a protrusion extending into the structural relief so that the protrusion is positioned in the drive recess with the coupling assembly in the advancing configuration and the protrusion is positioned in the securing recess with the coupling assembly in the rotary configuration, and the protrusion slides in the channel recess to shift the coupling assembly from the advancing configuration to the rotary configuration.

15. The surgical tool apparatus of claim 14, wherein the lock sleeve includes an opening defined on an end thereof having a profiled inner edge; the drive rod includes a protrusion on a proximate end thereof having a corresponding mating profile to be received in the lock sleeve opening; and with the coupling assembly in the rotary configuration, the protrusion being received in the lock sleeve opening so that the lock sleeve and drive rod rotate together.

16. The surgical tool apparatus of claim 1, further comprising a profiled end of the drive rod comprising a plurality of circumferentially spaced lobes configured to mate with a corresponding profile on the cap assembly at least one insert disposed between a pair of adjacent lobes, the insert permitting an interference fit with the cap assembly received on the profiled end of the drive rod.

17. A surgical tool apparatus for securing a rod and cap assembly into a coupling device, the surgical tool apparatus comprising:
a tubular body having a longitudinal axis;
a clamping mechanism for securely holding the coupling device;
a drive rod axially displaceable within the tubular body;
a single rotary handle operable to linearly advance the drive rod along the longitudinal axis and to rotate the drove rod about the longitudinal axis; and
a coupling device operable to shift between an advancing position where rotation of the handle advances the drive rod along the longitudinal axis and a rotary position where rotation of the handle rotates the drive rod about the longitudinal axis.

18. The surgical tool apparatus of claim 17, wherein the coupling device further includes:
a generally L-shaped slot defined in the elongate body
a lock bar arranged and configured to slide within a portion of the slot with the coupling assembly in the advancing position and to slide within another portion of the slot with the coupling assembly in the rotary position; and
wherein rotation of the handle automatically shifts the coupling assembly from the advancing position to the rotary position.

19. The surgical tool apparatus of claim 18, wherein coupling device includes an axial stop to limit movement of the drive rod along the longitudinal axis when a portion of the lock bar engages the axial stop and a rotary stop to limit rotational movement of the drive rod when another portion of the lock bar engages the rotary stop.

20. The surgical tool apparatus of claim 17, wherein the coupling device further includes:
a lock sleeve operable to rotate relative to the drive member with the coupling assembly in the advancing position and to rotate the drive rod with the coupling assembly in the rotary position; and
a position sleeve for shifting the lock sleeve from the advancing position to the rotary position.

21. The surgical tool apparatus of claim 20, wherein the position sleeve includes a guide member cooperating with the lock sleeve to shift the lock sleeve from the advancing position to the rotary position, and wherein the guide member includes a recess defined in an outer surface of the position sleeve and the lock sleeve includes a protrusion arranged and configured to slide within the recess.
